(19) United States
(12) Patent Application Publication
(10) Pub. No.: US 2022/0152138 A1
(43) Pub. Date: May 19, 2022

(54) METHODS FOR PRODUCTION OF LOW COST TERPENOIDS, INCLUDING CANNABINOIDS, AND VARIETIES ADAPTED FOR LARGE-SCALE PLANTING AND DENSITY OPTIMIZATION INCLUDING CANNABINOID PRESERVATION

(71) Applicant: NEW WEST GENETICS INC., Fort Collins, CO (US)

(72) Inventor: Richard S. Fletcher, Fort Collins, CO (US)

(21) Appl. No.: 17/594,513

(22) PCT Filed: May 8, 2020
(86) PCT No.: PCT/US2020/032047

§ 371 Date: Oct. 20, 2021

Related U.S. Application Data
(60) Provisional application No. 62/846,794, filed on May 13, 2019.
(60) Provisional application No. 62/845,383, filed on May 9, 2019.

(51) Int. Cl. A61K 36/185; A61K 9/00; A01G 22/60; A01D 45/00; A01C 7/08

(52) U.S. Cl. CPC A61K 36/185; A61K 9/0095; A01G 22/60; A01D 45/00; A61K 2236/35; A61K 2236/37; A61K 2236/331; A61K 2236/333; A01C 7/08

(57) ABSTRACT

According to the invention, there is provided novel hemp Cannabis cultivars with THC content of 0.2% by dry weight and a unique terpene profile. The cultivars exhibit increased CBD content of greater than about 1.07% by weight. The plants also have increased plant height and decreased branching that allows for increased yields of hemp and CBD content per acre. This invention thus relates to the seeds of hemp Cannabis cultivars of the invention, to the plants of hemp Cannabis cultivars of the invention, to plant parts of hemp Cannabis cultivars of the invention, to methods for producing a Cannabis cultivar by crossing the hemp Cannabis cultivars of the invention with another Cannabis cultivar, and to methods for producing a Cannabis cultivar containing in its genetic material one or more backcross conversion traits or transgenes and to the backcross conversion Cannabis plants and plant parts produced by those methods.

### CROSS-REFERENCE TO RELATED APPLICATIONS

This application claims priority to U.S. Provisional Application No. 62/845,383, filed May 9, 2019, and U.S. Provisional Application No. 62/846,794, filed May 13, 2019, the entire disclosures of which are expressly incorporated herein by reference for all purposes.

### FIELD OF THE INVENTION

The present invention relates to the field of plant breeding. In particular, this invention relates specialty cannabis plants, cultivars and varieties, including methods for making and using said cannabis plants and compositions derived thereof.

### BACKGROUND OF THE INVENTION

Industrial hemp is legally defined in the United States as Cannabis which contains 0.3% or less total sample dry weight of Δ9-Tetrahydrocannabinal (THC). THC content is normally well above the 0.30% threshold in modern varieties of Cannabis. THC is one of an estimated 85 cannabinoids (a class of terpenoids) synthesized in Cannabis species (El-Alfy et al., 2010, “Antidepressant-like effect of delta-9-tetrahydrocannabinol and other cannabinoids isolated from Cannabis sativa L”, Pharmacology Biochemistry and Behavior 95 (4): 434-42).

Cannabinoids act on endogenous cannabinoid receptors located throughout the human body (Kreitzer and Stella, 2009, “The therapeutic potential of novel cannabinoid receptors”, Pharmacology & Therapeutics 122 (2): 83-96). These receptors are present in humans because the human body manufactures a similar class of cannabinoids known as the endocannabinoids (Pertwee et al., 2010, “International Union of Basic and Clinical Pharmacology. LXXIX. Cannabinoid Receptors and Their Ligands: Beyond CB1 and CB2”, Pharmacological Reviews 62 (4): 588-631).

The demand for the medicinal properties of cannabinoids derived from Cannabis is growing. Over the last 15 years, medicinal marijuana has gained similar regulatory ground as hemp. This is a reflection of consumer demand. In 2013, medical marijuana sales were estimated at 1.5 billion dollars. The medicinal effects of cannabinoids on human health continue to be validated as clinical research in this field expands and gains traction (Scott et al., 2014, “The Combination of Cannabidiol and Δ9-Tetrahydrocannabinol Enhances the Anticancer Effects of Radiation in an Orthotopic Murine Glioma Model”, Molecular Cancer Therapeutics 13 (12): 2955-2967). The ability to create this medicine without THC is highly desired by many patients and regulatory agencies.

There is a large and growing market for cannabinoid products created from flower extracts. Many extraction methods exist and are designed to isolate compounds, thus creating concentrated products. In the case of hemp extraction, flower or chaff with ≤0.3% THC can be concentrated to levels ≥0.3%, thus disqualifying the extract as hemp. Additional steps to separate or extract THC from resultant purified cannabinoids to levels ≤0.3%, such as by reverse-phased flash chromatography, are often used to overcome this.

Terpenes are a large class of volatile organic hydrocarbons. In plants, they function as hormones (e.g. abscisic acid), as photosynthetic pigments (e.g. carotenoids) and are involved in many other vital physiological processes. Secondary terpenoids (secondary metabolites) account for the majority of terpenoid molecular structural diversity. The secondary terpenoids play a major role in the plant's response to environmental factors such as such as pathogen and photooxidative stresses (Tholl, 2006, “Terpene synthases and the regulation, diversity and biological roles of terpene metabolism”, Current Opinion in Plant Biology 9 (3): 297-304). Apart from their functions in the plant, terpenes from hops (Humulus lupulus) such as myrcene and humulene serve as major aromatic and flavor compounds in beer. Cannabis synthesizes many terpenes including myrcene and humulene. Cannabis normally reproduces under a dioecious system where male (staminate) and female (pistillate) flowers develop on separate plants. Monoecious plants (containing both male and female flowers) do exist. Female floral anatomy is characterized by pistils protruding from a calyx covered with resinous glandular trichomes. The glandular trichomes of the female flower are the primary site of cannabinoid synthesis. The female calyx contains ovaries and, therefore, is the site of seed development when fertilized by pollen produced by a male plant.

The Cannabis trichomes are rich in cannabinoids as well as terpenes. However, the trichomes are small (50-200 microns), fragile making them easily damaged by physical contact. Accordingly, growers typically handle Cannabis plants very carefully to limit trichome damage, loss and ultimately the reduction of cannabinoid content of the harvested product. This has constrained the use of mechanized harvesting, a necessary operation for large-scale commercial production.

A vast majority of the Cannabis produced in the United States is done so by clonal propagation. Under this production scheme, meristems are cut from a selected plant and treated by various methods to induce rooting so that many, genetically identical progeny may be derived from the original. This is primarily done because breeding Cannabis seeds which consistently express a particular cannabinoid profile, often elevated for a particular cannabinoid (e.g. THC), is generally regarded as difficult. The simplicity of breeding varieties to be produced under a clonal reproduction system is quickly offset by the cost of clonal production, among other factors (McKey et al., 2010, “The evolutionary ecology of clonally propagated domesticated plants”, New Phytologist 186 (2): 318-332). There is a need in the industry for industrial hemp varieties which are reliably low in THC when produced in diverse environmental conditions and which express elevated levels of certain other cannabinoids. The present invention provides a Cannabis variety that consistently and reproducibly has nearly zero THC (thus qualifying as industrial hemp) and elevated levels of CBD. Importantly the very low to zero THC in the varieties of the invention can avoid the additional separation steps often employed to reduce THC in extract products to below 0.3% THC.

Much of the production today is done using feminized seed with the end goal of fields containing only female plants. This is an intuitive approach since the female reproductive tissue (e.g. the buds) produce the economically-important cannabinoids and both approaches can control gender if they are done correctly. It also serves the purpose of eliminating, or at least minimizing, seed production which is perceived as important by many flower. The all-female production method comes with some notable tradeoffs:

High cost: clones and feminized seed are expensive to produce since this work is normally conducted in greenhouses and requires intensive management practices. Seed produced in greenhouses is of lower quality than field-produced seed resulting in lower germination rates. The cost of seed led producers to plant at lower population densities, ranging from 2,000 to 5,000 plants/acre in order to maximize the total yield per plant. These lower planting densities create the next tradeoff, weed control. Growers also have tended to germinate seeds indoors in trays to maximize germination rates which creates an extra cost and a more laborious planting process than direct seeding.

Poor weed control: commercial grain production is typically planted towards a goal of 500,000 plants per acres so that each acre of a dioecious cultivar contains around 250,000 female plants. This density follows traditional agronomy theory which seeks to minimize bare ground in order to control weeds more effectively. The low density of all-female production results in high weed control costs or a tolerance of a high weed seed bank in the following years.

Male pollen is difficult to control: hemp pollen is small and can be carried by wind for many miles (Small and Antle, 2003, “A Preliminary Study of Pollen Dispersal in Cannabis sativa in Relation to Wind Direction”, Journal of Industrial Hemp. 8(2): 37-50). This puts all-female hemp crops at risk of being pollinated by unknown fields with males, thus making the high input costs dubious. Further, most feminized seed providers do not guarantee 100% female seeds in their seed lots so walking fields to remove the rogue male is common practice and adds another production cost. It also presents a risk because even a single male plant can create tens of millions of pollen grains (Small and Antle, 2003, “A Preliminary Study of Pollen Dispersal in Cannabis sativa in Relation to Wind Direction”, Journal of Industrial Hemp. 8(2): 37-50). This means a single male plant can Feminized seed is just as it sounds, a seed source which produces predominantly female plants. A higher proportion of female plants is desirable since female plants are the source of almost all of the economically important products whether they be seed for grain, or flower (e.g. buds) for extraction.

Feminized seed is created by inducing female plants to become hermaphrodites via the application of chemicals like silverthiosulfate (Lubell and Brand, 2018, “Foliar sprays of silver thiosulfate produce male flowers and female hemp plants”, HortTechnology. 28(6): 743-747). Hermaphrodite plants will have female flowers which accept pollen to create seed, as well as male flowers which release pollen. The theory is that chemically-induced pollen created on a female plant contains the female sex chromosome (an X chromosome), rather than the male (Y chromosome). Thus, the progeny seed created by pollination with a hermaphrodite will inherit an X chromosome from the pollen and an X chromosome from the ovum of female flower. The resulting seeds will be primarily females but there is not yet a method which always produces 100% females. It is also known that pollen produced via this method is not of the same quality as that produced by true male hemp plants, which may also contribute to the aforementioned low germination rates of feminized seed (DiMatteo et al., 2020, “Pollen Appearance and In Vitro Germination Varies for Five Strains of Female Hemp Masculinized Using Silver Thiosulfate”, HortScience. 1-3).

There are numerous steps in the development of any novel, desirable plant germplasm. Plant breeding preferably begins with the analysis and definition of problems and weaknesses of the current germplasm, the establishment of program goals, and the definition of specific breeding objectives. The next step is preferable selection of germplasm that possess the traits to meet the program goals. The goal is to combine in a single variety or hybrid an improved combination of desirable traits from the parental germplasm.

### SUMMARY OF THE INVENTION

According to the invention, there is provided methods of increasing Cannabinoid yields from Cannabis. The methods comprise planting seeds from a Cannabis variety with an average CBD content of greater than about 1% by weight of female bud tissue, allowing seeds to grow into Cannabis plants with female buds, harvesting said Cannabis plants, and thereafter extracting CBD from said harvested Cannabis bud tissue. Preferably, the Cannabis is grown outdoors at large-scale commercial row crop production and provides reduced cost per acre relative to feminized transplant and feminized direct seeded models. In some aspects, the Cannabis is planted at a higher than average density to increase CBD yield per acre. Preferably, the seed is not feminized seed. In an aspect, the methods further comprise harvesting seed from the plant to further lower overall costs per acre.

According to the invention, there is provided novel hemp Cannabis varieties having very low levels of Δ9-Tetrahydrocanabinal (THC). The varieties exhibit on average less than about 0.2% THC. The varieties also demonstrate elevated levels of advantageous cannabinoids such as cannabidiol (CBD) and a ratio of CBD to THC of up to about 56:1. The varieties exhibit an average CBD content of greater than 1.07%, greater than 1.5%, greater than 2.0% greater than 2.5%, greater than 3%, greater than 4%, greater than 5%, or greater than 6% by weight and also have medium to short plant height with decreased branching that allow for higher density planting and increased levels of biomass (e.g. flower, chaff, grain, fiber, etc.) and CBD harvested per acre.

Further provided are Cannabis varieties having increased cannabinoid and/or trichome preservation. In an aspect, the varieties provide increased trichome preservation, including preservation from loss during mechanical harvesting. The trichomes of the Cannabis varieties are more resistant to loss during physical contact, heat, light, oxygen, and/or other sources of damage associated with outdoor production, thus resulting in higher cannabinoid yields (wt. %) after harvest using mechanical processes. Preferably at least 60%, at least 70%, at least 80%, at least 90%, at least 95%, at least 98%, or at least 99% of the trichomes are preserved during mechanized harvest enabling large-scale commercial production of cannabinoids. In another aspect, methods of assessing trichome and/or cannabinoid preservation of a Cannabis variety are provided. In some aspects, Cannabis varieties with increased trichome and/or cannabinoid preservation are selected for large-scale commercial row crop production based on the assessment.

This invention thus relates to the seeds of the hemp Cannabis cultivars of the invention, to plants of the hemp Cannabis cultivars of the invention, to plant parts of the hemp Cannabis cultivars of the invention, to methods for producing a Cannabis cultivar produced by crossing the hemp Cannabis cultivars of the invention with another Cannabis cultivar, and to methods for producing a Cannabis cultivar containing in its genetic material one or more backcross conversion traits or transgenes and to the backcross conversion Cannabis plants and plant parts produced by those methods.

This invention also relates to Cannabis cultivars and plant parts derived from the hemp Cannabis cultivars of the invention, to methods for producing other Cannabis cultivars derived from hemp Cannabis cultivars of the invention and to the Cannabis cultivars and their parts derived by the use of those methods. This invention further relates to Cannabis cultivar seeds, plants and plant parts produced by crossing the hemp Cannabis cultivars of the invention or a backcross conversion of the cultivars of the invention with another Cannabis cultivar.

The invention further relates to products and compositions produced or purified from plants of the invention including the stalks, fibers, pulp, flowers, seeds, hemp and the like. Products produced form the hemp cultivars of the invention can include industrial textiles, building materials, foods, personal hygiene products such as soap, lotions, balms and the like, animal bedding, industrial products such as paints, inks, solvents and lubricants, consumer textiles, animal feed, etc. The invention also relates to use of the Cannabis plants, plant parts extracts and the like as a flavoring or aromatic component in malt beverages and the like.

The presence of THC in extracts is particularly problematic and increases costs for foods (except grain), balms, animal feed, lotions, soap and hygiene products and malt beverages.

The invention includes varieties adapted for growing outside and at large scale to reduce costs and also for extraction of high value molecules (cannabinoids or terpenoids) from low THC plants to reduce downstream costs associated with removing THC.

### BRIEF DESCRIPTION OF THE DRAWINGS

FIGS. 1A-C show photos of resilient and sensitive trichomes. FIG. 1A shows a fresh bract with trichomes mostly intact. FIG. 1B shows a resilient post-threshing example with many intact trichomes. FIG. 1C shows a sensitive post-threshing example with trichome heads largely lost.

FIG. 2 shows average trichome retention of four high CBD genotypes versus four low CBD genotypes.

FIG. 3 shows differences in cannabinoid loss after gentle or aggressive threshing methods in 43 genotypes. Exemplary susceptible (2833) and resilient (2909) genotypes are shown as a dashed black line and a solid black line, respectively.

FIG. 4 shows trichome retention of aggressively threshed field samples of 2909 and 2833.

FIGS. 5A-D show the financials of producing CBD with different models. FIG. 5A shows the row crop model with a low CBD (2.8%) variety. FIG. 5B shows the row crop model with a high CBD (6.2%) variety. FIG. 5C shows the feminized transplant model. FIG. 5D shows the feminized direct seeded model. Key differences are indicated in bold italics.

### DETAILED DESCRIPTION OF THE INVENTION

### Definitions

In the description and tables which follow, a number of terms are used. In order to provide a clear and consistent understanding of the present invention, the following definitions are provided:

The invention provides cannabis plants. As used herein, the term “plant” refers to plants in the genus of Cannabis and plants derived thereof. Such as cannabis plants produced via asexual reproduction and via seed production.

The invention provides plant parts. As used herein, the term “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”. Plant part may also include certain extracts such as kief or hash which includes cannabis trichomes or glands.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, a “landrace” refers to a local variety of a domesticated plant species which has developed largely by natural processes, by adaptation to the natural and cultural environment in which it lives. The development of a landrace may also involve some selection by humans, but it differs from a formal breed which has been selectively bred deliberately to conform to a particular formal, purebred standard of traits.

The invention provides plant cultivars. As used herein, the term “cultivar” means a group of similar plants that by structural features and performance (i.e., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain and race are often used interchangeably by plant breeders, agronomists and farmers.

As used herein, the term “elite line” or “elite germplasm” means any line, cultivar or variety that has resulted from breeding and selection for superior agronomic performance. An elite plant is any plant from an elite line or elite germplasm.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

As used herein, the term “inbreeding” refers to the production of offspring via the mating between relatives. The plants resulting from the inbreeding process are referred to herein as “inbred plants” or “inbreds.”

The term LOQ as used herein refers to the limit of quantitation for Gas Chromatography (GC) and High-Performance Liquid Chromatography measurements.

The term secondary metabolites as used herein refers to organic compounds that are not directly involved in the normal growth, development, or reproduction of an organism. In other words, loss of secondary metabolites does not result in immediate death of said organism.

The term single allele converted plant as used herein refers to those plants which are developed by a plant breeding technique called backcrossing wherein essentially all of the desired morphological and physiological characteristics of an inbred are recovered in addition to the single allele transferred into the inbred via the backcrossing technique.

The invention provides samples. As used herein, the term “sample” includes a sample from a plant, a plant part, a plant cell, or from a transmission vector, or a soil, water or air sample.

The invention provides progeny. As used herein, the term “progeny” refers to any plant resulting from a vegetative or sexual reproduction from one or more parent plants or descendants thereof. For instance, a progeny plant may be obtained by cloning or selfing of a parent plant or by crossing two parent plants and include selfings as well as the F1 or F2 or still further generations. An F1 is a first-generation progeny produced from parents at least one of which is used for the first time as donor of a trait, while offspring of second generation (F2) or subsequent generations (F3, F4, etc.) are specimens produced from selfings of F1's F2's etc. An F1 may thus be (and usually is) a hybrid resulting from a cross between two true breeding parents (true-breeding is homozygous for a trait), while an F2 may be (and usually is) a progeny resulting from self-pollination of said F1 hybrids.

The invention provides methods for crossing a first plant with a second plant. As used herein, the term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first-generation hybrid (F1), back to one of the parents of the hybrid progeny. Backcrossing can be used to introduce one or more single locus conversions from one genetic background into another.

The term backcrossing is a process in which a breeder crosses progeny back to one of the parents one or more times, for example, a first-generation hybrid F₁ with one of the parental genotypes of the F₁ hybrid.

The invention provides donor plants and recipient plants. As used herein, “donor plants” refer to the parents of a variety which contains the gene or trait of interest which is desired to be introduced into a second variety (e.g., “recipient plants”).

In some embodiments, the present invention provides methods for obtaining plant genotypes comprising recombinant genes. As used herein, the term “genotype” refers to the genetic makeup of an individual cell, cell culture, tissue, organism (e.g., a plant), or group of organisms.

In some embodiments, the present invention provides homozygotes. As used herein, the term “homozygote” refers to an individual cell or plant having the same alleles at one or more loci.

In some embodiments, the present invention provides homozygous plants. As used herein, the term “homozygous” refers to the presence of identical alleles at one or more loci in homologous chromosomal segments.

In some embodiments, the present invention provides hemizygotes. As used herein, the term “hemizygotes” or “hemizygous” refers to a cell, tissue, organism or plant in which a gene is present only once in a genotype, as a gene in a haploid cell or organism, a sex-linked gene in the heterogametic sex, or a gene in a segment of chromosome in a diploid cell or organism where its partner segment has been deleted.

In some embodiments, the present invention provides heterozygotes. As used herein, the terms “heterozygote” and “heterozygous” refer to a diploid or polyploid individual cell or plant having different alleles (forms of a given gene) present at least at one locus. In some embodiments, the cell or organism is heterozygous for the gene of interest which is under control of the synthetic regulatory element.

The invention provides methods for obtaining plant lines comprising recombinant genes. As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The invention provides open-pollinated populations. As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

The invention provides self-pollination populations. As used herein, the term “self-crossing”, “self-pollinated” or “self-pollination” means the pollen of one flower on one plant is applied (artificially or naturally) to the ovule (stigma) of the same or a different flower on the same plant.

The invention provides ovules and pollens of plants. As used herein when discussing plants, the term “ovule” refers to the female gametophyte, whereas the term “pollen” means the male gametophyte.

The invention provides plant tissue. As used herein, the term “plant tissue” refers to any part of a plant. Examples of plant organs include, but are not limited to the trichome, leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The invention provides methods for obtaining plants comprising recombinant genes through transformation. As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

The invention provides transformants comprising recombinant genes. As used herein, the term “transformant” refers to a cell, tissue or organism that has undergone transformation. The original transformant is designated as “T0” or “T₀.” Selfing the T0 produces a first transformed generation designated as “T1” or “T₁.”

In some embodiments, the present invention provides organisms with recombinant genes. As used herein, an “organism” refers any life form that has genetic material comprising nucleic acids including, but not limited to, prokaryotes, eukaryotes, and viruses. Organisms of the present invention include, for example, plants, animals, fungi, bacteria, and viruses, and cells and parts thereof.

As used herein, the term “female” refers to Cannabis plants carrying only pistillate flowers and devoid of pollen. The term “bud” refers to Cannabis female floral tissue collected prior to seed harvest from the apical meristems. The term “chaff” refers to Cannabis bud tissue collected after threshing and separation of physiologically mature seed from the bud. The term “male” refers to Cannabis plants carrying only staminate flowers producing pollen.

In embodiments of the invention the level of CBD content is at least 1.07% or more based upon total dry weight of the plant and in further embodiments is at least 1.5%, 2%, 2.5%, 3%, 3.5%, 4%, 4.5%, 5%, 5.5%, 6%, 6.5%, 7%, 7.5%, 8%, 8.5%, 9%, 10% or more or amounts in-between of CBD content based on total dry weight of the plant.

### Cannabis

Cannabis has long been used for drug and industrial purposes including fiber, seed and seed oils, and for medicinal purposes. Industrial hemp fiber products are made from Cannabis plants selected to produce an abundance of stalk tissue from which fiber is created.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the cannabis plant (Rudolf Brenneisen, 2007, Chemistry and Analysis of Phytocannabinoids (cannabinoids produced by cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). The two cannabinoids usually produced in greatest abundance are cannabidiol (CBD) and/or Δ-9-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

The cannabinoids in cannabis plants include, but are not limited to, A 9 Tetrahydrocannabinol (.Δ9-THC), Δ. 8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ.9-Tetrahydrocannabivarin (THCV), cannabichromevarin (CBCV), and cannabigerovarin (CBGV). See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

### Cannabis Chemistry

Cannabinoids are a class of diverse chemical compounds that activate cannabinoid receptors of the human endocannabinoid physiological system. Cannabinoids produced by plants are called phytocannabinoids, a.k.a., natural cannabinoids, herbal cannabinoids, and classical cannabinoids. At least 85 different cannabinoids have been isolated from the cannabis plants (El-Alfy et al., 2010, “Antidepressant-like effect of delta-9-tetrahydrocannabinol and other cannabinoids isolated from Cannabis sativa L”, Pharmacology Biochemistry and Behavior 95 (4): 434-42; Brenneisen, supra). Typical cannabinoids isolated from cannabis plants include, but are not limited to, Tetrahydrocannabinol (THC), Cannabidiol (CBD), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), and CBGM (Cannabigerol Monomethyl Ether). In the Cannabis plant, cannabinoids are synthesized and accumulated as cannabinoid acids (e.g., cannabidiolic acid (CBDA)). When the herbal product is dried, stored, or heated, the acids decarboxylize gradually or completely into neutral forms (e.g., CBDA→CBD).

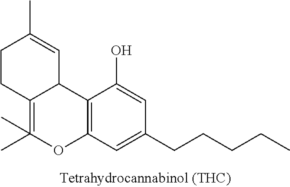

Known as delta-9-tetrahydrocannabinol (Δ9-THC), THC is the principal psychoactive constituent (or cannabinoid) of the cannabis plant. The initially synthesized and accumulated form in plant is THC acid (THCA).

THC has mild to moderate analgesic effects, and Cannabis can be used to treat pain by altering transmitter release on dorsal root ganglion of the spinal cord and in the periaqueductal gray. Other effects include relaxation, alteration of visual, auditory, and olfactory senses, fatigue, and appetite stimulation. THC has marked antiemetic properties and may also reduce aggression in certain subjects (Hoaken (2003). “Drugs of abuse and the elicitation of human aggressive behavior”. Addictive Behaviors 28: 1533-1554).

The pharmacological actions of THC result from its partial agonist activity at the cannabinoid receptor CB1, located mainly in the central nervous system, and the CB2receptor, mainly expressed in cells of the immune system (Pertwee, 2006, “The pharmacology of cannabinoid receptors and their ligands: An overview”. International Journal of Obesity 30: S13-S18.) The psychoactive effects of THC are primarily mediated by its activation of CB1G-protein coupled receptors, which result in a decrease in the concentration of the second messenger molecule cAMP through inhibition of adenylate cyclase (Elphick et al., 2001, “The neurobiology and evolution of cannabinoid signaling”. Philosophical Transactions of the Royal Society B: Biological Sciences 356 (1407): 381-408.) It is also suggested that THC has an anticholinesterase action which may implicate it as a potential treatment for Alzheimer's and Myasthenia (Eubanks et al., 2006, “A Molecular Link Between the Active Component of Marijuana and Alzheimer's Disease Pathology”. Molecular Pharmaceutics 3 (6): 773-7.) In the cannabis plant, THC occurs mainly as tetrahydrocannabinolic acid (THCA, 2-COOH-THC). Geranyl pyrophosphate and olivetolic acid react, catalyzed by an enzyme to produce cannabigerolic acid, which is cyclized by the enzyme THC acid synthase to give THCA. Over time, or when heated, THCA is decarboxylated to produce THC. The pathway for THCA biosynthesis is similar to that which produces the bitter acid humulone in hops. See Fellermeier et al., (1998, “Prenylation of olivetolate by a hemp transferase yields cannabigerolic acid, the precursor of tetrahydrocannabinol”. FEBS Letters 427 (2): 283-5); de Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112.)

CBD is a cannabinoid found in cannabis. Cannabidiol has displayed sedative effects in animal tests (Pickens, 1981, “Sedative activity of cannabis in relation to its delta′-trans-tetrahydrocannabinol and cannabidiol content”. Br. J. Pharmacol. 72 (4): 649-56). Some research, however, indicates that CBD can increase alertness, and attenuate the memory-impairing effect of THC. (Nicholson et al., June 2004, “Effect of Delta-9-tetrahydrocannabinol and cannabidiol on nocturnal sleep and early-morning behavior in young adults” J Clin Psychopharmacol 24 (3): 305-13; Morgan et al., 2010, “Impact of cannabidiol on the acute memory and psychotomimetic effects of smoked cannabis: naturalistic study, The British Journal of Psychiatry, 197:258-290). It may decrease the rate of THC clearance from the body, perhaps by interfering with the metabolism of THC in the liver. Medically, it has been shown to relieve convulsion, inflammation, anxiety, and nausea, as well as inhibit cancer cell growth (Mechoulam, et al., 2007, “Cannabidiol—recent advances”. Chemistry & Biodiversity 4 (8): 1678-1692.) Recent studies have shown cannabidiol to be as effective as atypical antipsychotics in treating schizophrenia (Zuardi et al., 2006, “Cannabidiol, a Cannabis sativa constituent, as an antipsychotic drug” Braz. J. Med. Biol. Res. 39 (4): 421-429.). Studies have also shown that it may relieve symptoms of dystonia (Consroe, 1986, “Open label evaluation of cannabidiol in dystonic movement disorders”. The International journal of neuroscience 30 (4): 277-282). CBD reduces growth of aggressive human breast cancer cells in vitro and reduces their invasiveness (McAllister et al., 2007, “Cannabidiol as a novel inhibitor of Id-1 gene expression in aggressive breast cancer cells”. Mol. Cancer. Ther. 6 (11): 2921-7.)

Cannabidiol has shown to decrease activity of the limbic system (de Souza Crippa et al., “Effects of Cannabidiol (CBD) on Regional Cerebral Blood Flow”. Neuropsychopharmacology 29 (2): 417-426.) and to increase social interaction which is often decreased by THC (Malon et al., “Cannabidiol reverses the reduction in social interaction produced by low dose Δ9-tetrahydrocannabinol in rats”. Pharmacology Biochemistry and Behavior 93 (2): 91-96.) It's also shown that Cannabidiol reduces anxiety in social anxiety disorder (Bergamaschi et al., 2003, “Cannabidiol Reduces the Anxiety Induced by Simulated Public Speaking in Treatment-Naive Social Phobia Patients”. Neuropsychopharmacology 36 (6): 1219-1226). Cannabidiol has also been shown as being effective in treating an often drug-induced set of neurological movement disorders known as dystonia (Snider et al., 1985, “Beneficial and Adverse Effects of Cannabidiol in a Parkinson Patient with Sinemet-Induced Dystonic Dyskinesia”. Neurology, (Suppl 1): 201.) Morgan et al. reported that strains of cannabis which contained higher concentrations of Cannabidiol did not produce short-term memory impairment vs. strains which contained similar concentrations of THC (2010, “Impact of cannabidiol on the acute memory and psychotomimetic effects of smoked cannabis: naturalistic study: naturalistic study [corrected.”]. British Journal of Psychiatry 197 (4): 285-90.)

Cannabidiol acts as an indirect antagonist of cannabinoid agonists. CBD is an antagonist at the putative new cannabinoid receptor, GPR55. Cannabidiol has also been shown to act as a 5-HT1A receptor agonist, an action which is involved in its antidepressant, anxiolytic, and neuroprotective effects. Cannabidiol is also an allosteric modulator at the Mu and Delta opioid receptor sites.

Cannabis produces CBD-carboxylic acid through the same metabolic pathway as THC, until the last step, where CBDA synthase performs catalysis instead of THCA synthase. See Marks et al. (2009, “Identification of candidate genes affecting Δ9-tetrahydrocannabinol biosynthesis in Cannabis sativa”. Journal of Experimental Botany 60 (13): 3715-3726.) and Meijer et al. I, II, III, and IV. Non-limiting examples of CBD variants include:

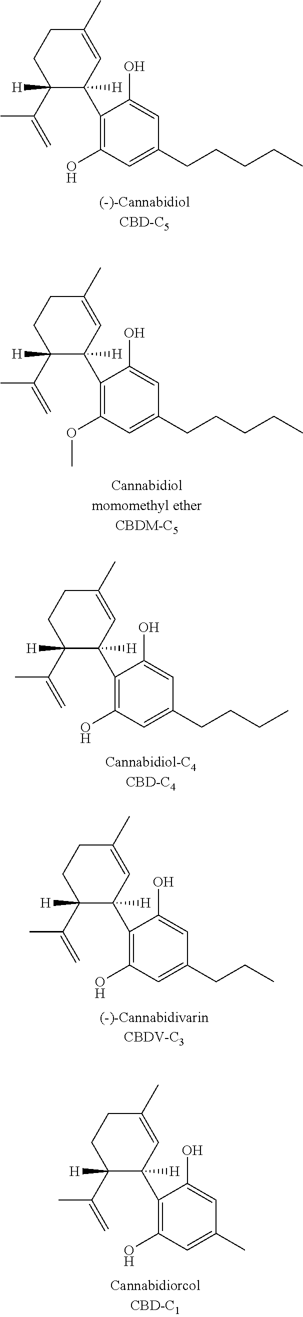

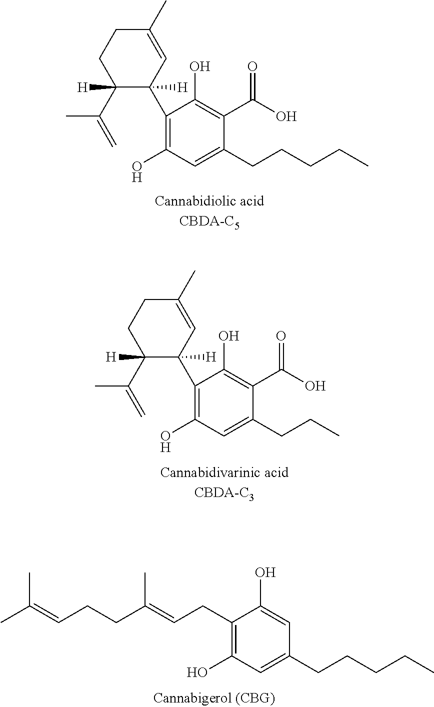

CBG is a non-psychoactive cannabinoid found in the Cannabis genus of plants. Cannabigerol is found in higher concentrations in hemp rather than in varieties of Cannabis cultivated for high THC content and their corresponding psychoactive properties. Cannabigerol has been found to act as a high affinity α-2-adrenergic receptor agonist, moderate affinity 5-HT1A receptor antagonist, and low affinity CB.sub.1 receptor antagonist. It also binds to the CB2receptor. Cannabigerol has been shown to relieve intraocular pressure, which may be of benefit in the treatment of glaucoma (Craig et al. 1984, “Intraocular pressure, ocular toxicity and neurotoxicity after administration of cannabinol or cannabigerol” Experimental eye research 39 (3):251-259). Cannabigerol has also been shown to reduce depression in animal models (U.S. patent application Ser. No. 11/760,364). Non-limiting examples of CBG variants include:

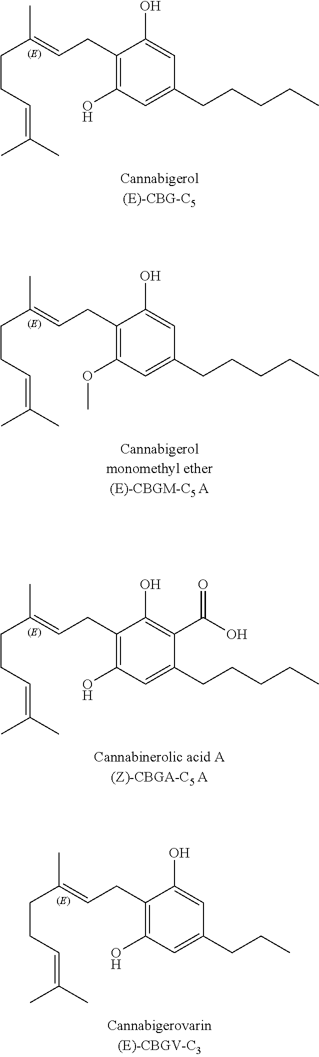 [SMILES: CCCCCc1cc(O)c(C/C=C(\C)CCC=C(C)C)c(O)c1 ; CCCCCc1cc(O)c(C/C=C(\C)CCC=C(C)C)c(OC)c1 ; CCCCCc1cc(O)c(C/C=C(\C)CCC=C(C)C)c(O)c1C(=O)O ; CCCc1cc(O)c(C/C=C(\C)CCC=C(C)C)c(O)c1]

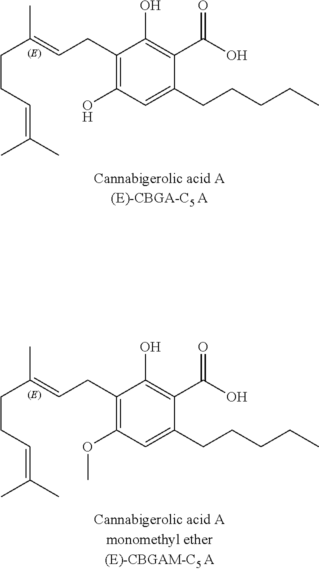 [SMILES: CCCCCc1cc(O)c(C/C=C(\C)CCC=C(C)C)c(O)c1C(=O)O ; CCCCCc1cc(OC)c(C/C=C(\C)CCC=C(C)C)c(O)c1C(=O)O]

CBN is a psychoactive substance cannabinoid found in Cannabis sativa and Cannabis indica/afghanica. It is also a metabolite of tetrahydrocannabinol (THC). CBN acts as a weak agonist of the CB1 and CB2 receptors, with lower affinity in comparison to THC.

CBC bears structural similarity to the other natural cannabinoids, including tetrahydrocannabinol, tetrahydrocannabivarin, cannabidiol, and cannabinol, among others. Evidence has suggested that it may play a role in the anti-inflammatory and anti-viral effects of cannabis and may contribute to the overall analgesic effects of cannabis. Non-limiting examples of CBC variants include:

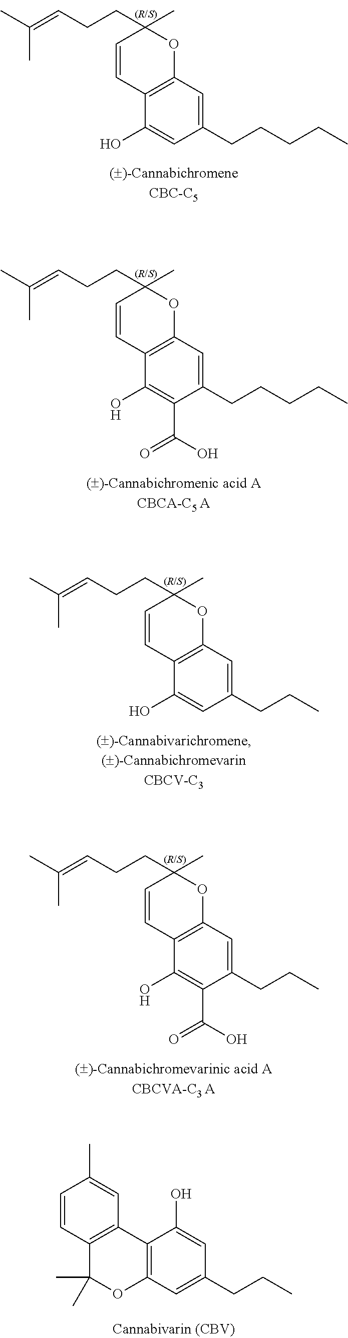

Cannabivarin, also known as cannabivarol or CBV, is a non-psychoactive cannabinoid found in minor amounts in the hemp plant Cannabis sativa. It is an analog of cannabinol (CBN) with the side chain shortened by two methylene bridges (—CH2-). CBV is an oxidation product of tetrahydrocannabivarin (THCV, THV).

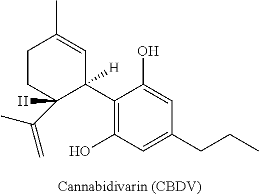 [SMILES: C=C(C)[C@@H]1CCC(C)=C[C@H]1c1c(O)cc(CCC)cc1O]

CBDV is a non-psychoactive cannabinoid found in Cannabis. It is a homolog of cannabidiol (CBD), with the side-chain shortened by two methylene bridges (CH2 units). Cannabidivarin has been found reduce the number and severity of seizures in animal models (U.S. patent application Ser. No. 13/075,873). Plants with relatively high levels of CBDV have been reported in feral populations of C. indica (=C. sativa ssp. indica var. kafiristanica) from northwest India, and in hashish from Nepal.

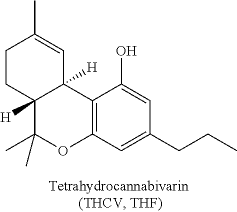 [SMILES: CCCc1cc(O)c2c(c1)OC(C)(C)[C@@H]1CCC(C)=C[C@@H]21]

THCV, or THV is a homologue of tetrahydrocannabinol (THC) having a propyl (3-carbon) side chain. This terpeno-phenolic compound is found naturally in Cannabis, sometimes in significant amounts. Plants with elevated levels of propyl cannabinoids (including THCV) have been found in populations of Cannabis sativa L. ssp. indica (=Cannabis indica Lam.) from China, India, Nepal, Thailand, Afghanistan, and Pakistan, as well as southern and western Africa. THCV has been shown to be a CB1 receptor antagonist, i.e. it blocks the effects of THC. Tetrahydrocannabinol has been shown to increase metabolism, help weight loss and lower cholesterol in animal models (U.S. patent application Ser. No. 11/667,860).

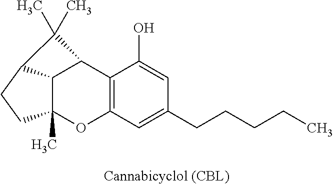

Cannabicyclol (CBL) is a non-psychotomimetic cannabinoid found in the Cannabis species. CBL is a degradative product like cannabinol. Light converts cannabichromene to CBL. Non-limiting examples of CBL variants include:

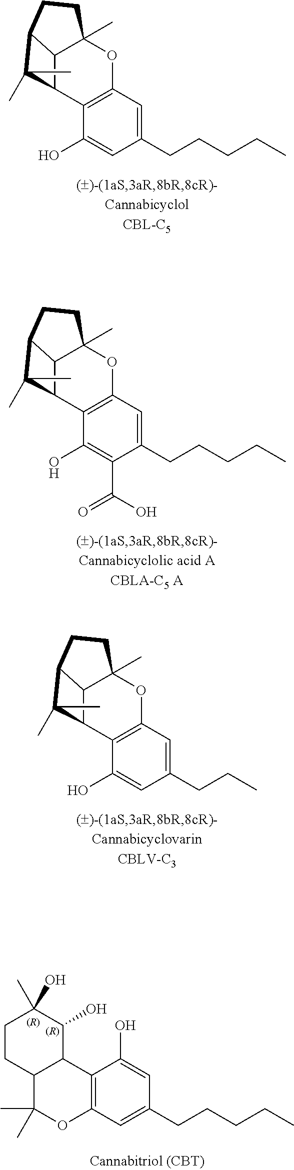

Non-limiting examples of CBT variants include:

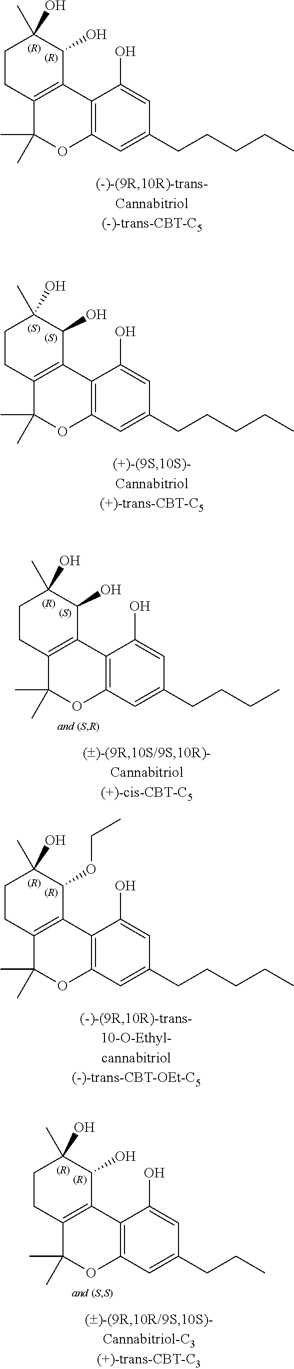

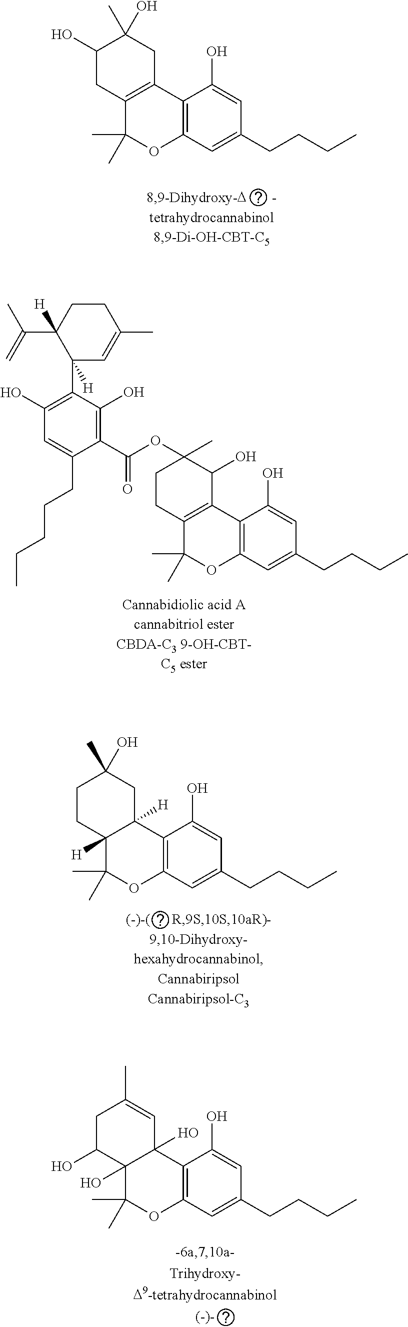

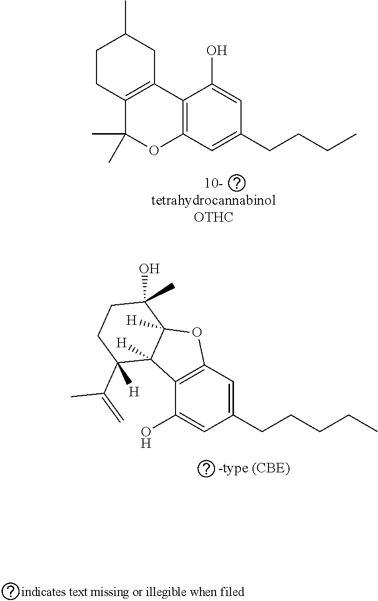

Non limiting examples of CBE variants include:

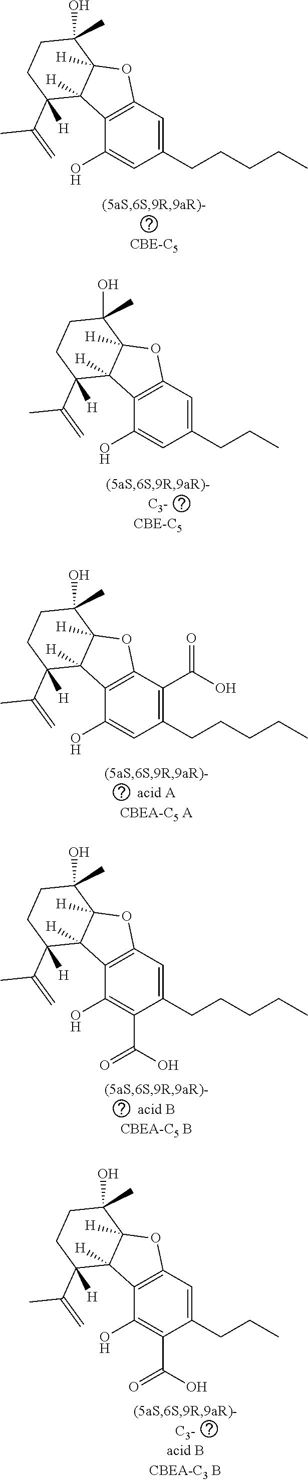

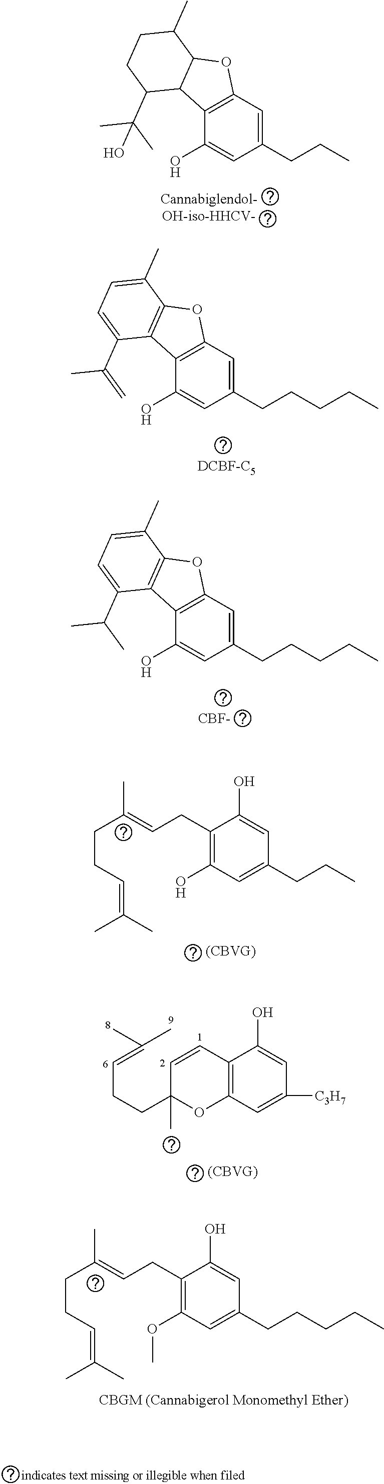

The biosynthetic pathway of cannabinoids has been studied. See Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112), each of which is herein incorporated by reference in its entirety for all purposes. According to the current model, phenolic precursors such as geranyl pyrophosphate (GPP) and polyketide, olivetolic acid (OA) are condensed by geranyl pyrophosphate olivetolate geranyltransferase (GOT) to form Cannabigerol acid (CBGA). Alternatively, GPP and divarine acid are condensed by GOT to form Cannabigerovarinic acid (CBGVA). CBGA or CBGAV is transformed to (1) CBC by CBC synthase or CBCV by CBCV synthase; (2) THC by THC synthase or THCV by THCV synthase; or (3) CBD by CBD synthase or CBDV by CBDV synthase. The genes coding for THC synthase and CBD synthase are found on the same B locus. Thus cannabis plants can be categorized into THC-CBD chemotypes based on the state of the B locus B_{T}/B_{T} (THC producing, chemotype B_{D}/B_{D} (CBD producing, chemotype III), and B_{T}/B_{D} (producing both THC and CBD, chemotype II). Additional information on the genetic regulation of cannabinoids can be found in Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112).

More details of cannabinoids synthesis and the properties and uses of these cannabinoids are described in Russo (2011, Taming THC: potential cannabis synergy and phytocannabinoid-terpenoid entourage effects, British Journal of Pharmacology, 163:1344-1364), Russo et al. (2006, A tale of two cannabinoids: the therapeutic rationale for combining tetrahydrocannabinol and cannabidiol, Medical Hypothesis, 2006, 66:234-246), Celia et al. (Impact of cannabidiol on the acute memory and psychotomimetic effects of smoked cannabis: naturalistic study, The British Journal of Psychiatry, 201, 197:285-290), de Mello Schier et al., (Cannabidiol, a Cannabis sativa constituent, as an anxiolytic drug, Rev. Bras. Psiquiatr, 2012, 34(S1):5104-5117), and Zhornitsky et al. (Cannabidiol in Humans—the Quest for Therapeutic Targets, Pharmaceuticals, 2012, 5:529-552), and U.S. Pat. No. 9,095,554, each of which is herein incorporated by reference in its entirety for all purposes.

### Terpenes and Terpenoids in Cannabis Plants

Terpenes are a large and diverse class of organic compounds, produced by Cannabis plants. They are often strong smelling and thus may have had a protective function. Terpenes are derived biosynthetically from units of isoprene, which has the molecular formula C₅.H₈. The basic molecular formulae of terpenes are multiples of that, (C₅H₈)ₙ where n is the number of linked isoprene units. The isoprene units may be linked together “head to tail” to form linear chains or they may be arranged to form rings. Non-limiting examples of terpenes include Hemiterpenes, Monoterpenes, Sesquiterpenes, Diterpenes, Sesterterpenes, Triterpenes, Sesquarterpenes, Tetraterpenes, Polyterpenes, and Norisoprenoids.

Terpenoids, a.k.a. isoprenoids, are a large and diverse class of naturally occurring organic chemicals similar to terpenes, derived from five-carbon isoprene units assembled and modified in thousands of ways. Most are multicyclic structures that differ from one another not only in functional groups but also in their basic carbon skeletons. Plant terpenoids are used extensively for their aromatic qualities. They play a role in traditional herbal remedies and are under investigation for antibacterial, antineoplastic, and other pharmaceutical functions. The terpene Linalool for example, has been found to have anti-convulsant properties (Elisabetsky et al., Phytomedicine, May 6(2):107-13 1999). Well-known terpenoids include citral, menthol, camphor, salvinorin A in the plant Salvia divinorum, and the cannabinoids found in Cannabis. Non-limiting examples of terpenoids include, Hemiterpenoids, 1 isoprene unit (5 carbons); Monoterpenoids, 2 isoprene units (10C); Sesquiterpenoids, 3 isoprene units (15C); Diterpenoids, 4 isoprene units (20C) (e.g. ginkgolides); Sesterterpenoids, 5 isoprene units (25C); Triterpenoids, 6 isoprene units (30C) (e.g. sterols); Tetraterpenoids, 8 isoprene units (40C) (e.g. carotenoids); and Polyterpenoid with a larger number of isoprene units.

In addition to cannabinoids, Cannabis also produces over 120 different terpenes (Russo 2011, Taming THC: potential cannabis synergy and phytocannabinoid-terpenoid entourage effects, British Journal of Pharmacology, 163:1344-1364). Within the context and verbiage of this document the terms ‘terpenoid’ and ‘terpene’ are used interchangeably. Cannabinoids are odorless, so terpenoids are responsible for the unique odor of Cannabis, and each variety has a slightly different profile that can potentially be used as a tool for identification of different varieties or geographical origins of samples (Hillig 2004. “A chemotaxonomic analysis of terpenoid variation in Cannabis” Biochem System and Ecology 875-891). It also provides a unique and complex organoleptic profile for each variety that is appreciated by both novice users and connoisseurs. In addition to many circulatory and muscular effects, some terpenes interact with neurological receptors. A few terpenes produced by Cannabis plants also bind weakly to Cannabinoid receptors. Some terpenes can alter the permeability of cell membranes and allow in either more or less THC, while other terpenes can affect serotonin and dopamine chemistry as neurotransmitters. Terpenoids are lipophilic, and can interact with lipid membranes, ion channels, a variety of different receptors (including both G-protein coupled odorant and neurotransmitter receptors), and enzymes. Some are capable of absorption through human skin and passing the blood brain barrier.

Generally speaking, terpenes are considered to be pharmacologically relevant when present in concentrations of at least 0.05% in plant material (Hazekamp and Fischedick 2010. “Metabolic fingerprinting of Cannabis sativa L., cannabinoids and terpenoids for chemotaxonomic and drug standardization purposes” Phytochemistry 2058-73; Russo 2011, Taming THC: potential cannabis synergy and phytocannabinoid-terpenoid entourage effects, British Journal of Pharmacology, 163:1344-1364). Thus, although there are an estimated 120 different terpenes, only a few are produced at high enough levels to be detectable, and fewer still which are able to reach pharmacologically relevant levels.

A Cannabis terpene profile is includes the absolute and relative values of the 25 of the most measured terpenes disclosed herein, including but not limited to: terpinolene, alpha phellandrene, beta ocimene, carene, limonene, gamma terpinene, alpha pinene, alpha terpinene, beta pinene, camphene, alpha terpineol, alpha humulene, beta caryophyllene, linalool, caryophyllene oxide, and myrcene. Both experts and consumers believe that there are biochemical and phenomenological differences between different varieties of cannabis, which are attributed to their unique relative cannabinoid and terpenoid ratios. This is known as the entourage effect and is generally considered to result in plants providing advantages over only using the natural products that are isolated from them (Russo 2011, Taming THC: potential cannabis synergy and phytocannabinoid-terpenoid entourage effects, British Journal of Pharmacology, 163:1344-1364).

Terpenoids can be extracted from the plant material by steam distillation (giving you essential oil) or vaporization, however the yield varies greatly by plant tissue, type of extraction, age of material, and other variables (McPartland and Russo 2001 “Cannabis and Cannabis Extracts: Greater Than the Sum of Their Parts?” Hayworth Press). Typically, the yield of terpenoids in Cannabis is less than 1% by weight on analysis; however, it is thought that they may comprise up to 10% of the trichome content. Monoterpenoids are especially volatile, thus decreasing their yield relative to sesquiterpenoids (Russo 2011, Taming THC: potential cannabis synergy and phytocannabinoid-terpenoid entourage effects, British Journal of Pharmacology, 163:1344-1364).

D-Limonene is a monoterpenoid that is widely distributed in nature and often associated with citrus. It has strong anxiolytic properties in both mice and humans, apparently increasing serotonin and dopamine in mouse brain. D-limonene has potent anti-depressant activity when inhaled. It is also under investigation for a variety of different cancer treatments, with some focus on its hepatic metabolite, perillic acid. There is evidence for activity in the treatment of dermatophytes and gastro-oesophageal reflux, as well as having general radical scavenging properties (Russo 2011, Taming THC: potential cannabis synergy and phytocannabinoid-terpenoid entourage of British Journal of Pharmacology, 163:1344-1364).

β-Myrcene is a monoterpenoid also found in cannabis and has a variety of pharmacological effects. It is often associated with a sweet fruit like taste. It reduces inflammation, aids sleep, and blocks hepatic carcinogenesis, as well as acting as an analgesic and muscle relaxant in mice. When βmyrcene is combined with Δ9-THC it could intensify the sedative effects of Δ9-THC, causing the well-known “couch-lock” effect that some Cannabis users experience (Russo 2011, Taming THC: potential cannabis synergy and phytocannabinoid-terpenoid entourage effects, British Journal of Pharmacology, 163:1344-1364).

D-Linalool is a monoterpenoid with very well-known anxiolytic effects. It is often associated with lavender, and frequented used in aromatherapy for its sedative impact. It acts as a local anaesthetic and helps to prevent scarring from burns, is anti-nociceptive in mice, and shows antiglutamatergic and anticonvulsant activity. Its effects on glutamate and GABA neurotransmitter systems are credited with giving it its sedative, anxiolytic, and anticonvulsant activities (Russo 2011, Taming THC: potential cannabis synergy and phytocannabinoid-terpenoid entourage effects, British Journal of Pharmacology, 163:1344-1364).

α-Pinene is a monoterpene common in nature, also with a plethora of effects on mammals and humans. It acts as an acetylcholinesterase inhibitor which aids memory and counteracts the short-term memory loss associated with Δ9-THC intoxication, is an effective antibiotic agent, and shows some activity against MRSA. In addition, α-pinene is a bronchodilator in humans and has anti-inflammatory properties via the prostaglandin E-1 pathway (Russo 2011, Taming THC: potential cannabis synergy and phytocannabinoid-terpenoid entourage effects, British Journal of Pharmacology, 163:1344-1364).

β-Caryophyllene is often the most predominant sesquiterpenoid in cannabis. It is less volatile than the monoterpenoids, thus it is found in higher concentrations in material that has been processed by heat to aid in decarboxylation. It is very interesting in that it is a selective full agonist at the CB2 receptor, which makes it the only phytocannabinoid found outside the cannabis genus. In addition, it has anti-inflammatory and gastric cytoprotective properties, and may even have anti-malarial activity.

Caryophyllene oxide is another sesquiterpenoid found in cannabis, which has antifungal and anti-platelet aggregation properties. As an aside, it is also the molecule that drug-sniffing dogs are trained to find (Russo 2011, Taming THC: potential cannabis synergy and phytocannabinoid-terpenoid entourage effects, British Journal of Pharmacology, 163:1344-1364).

Nerolidol is a sesquiterpene that is often found in citrus peels that exhibits a range of interesting properties. It acts as a sedative, inhibits fungal growth, and has potent anti-malarial and antileishmanial activity. It also alleviated colon adenomas in rats (Russo 2011, Taming THC: potential cannabis synergy and phytocannabinoid-terpenoid entourage effects, British Journal of Pharmacology, 163:1344-1364). Phytol is a diterpene often found in cannabis extracts. It is a degradation product of chlorophyll and tocopherol. It increases GABA expression and therefore could be responsible the relaxing effects of green tea and wild lettuce. It also prevents vitamin-A induced teratogenesis by blocking the conversion of retinol to its dangerous metabolite, all-trans-retinoic acid (Russo 2011, Taming THC: potential cannabis synergy and phytocannabinoid-terpenoid entourage effects, British Journal of Pharmacology, 163:1344-1364).

### Methods of Extraction

CO2 Extraction is one such method which takes advantage of the differing solubility of waxes and cannabinoids at temperatures below the freezing point. Essentially, the frozen wax from the hemp plant containing cannabidiol oil is stripped from the plant using carbon dioxide. The wax is then separated from the solvent using a filter press.

Solvent Extraction is another common method which utilizes a maceration process where ground-up hemp is mixed with a solvent such as ethanol. The solvent strips the CBD oil out of the plant fibers, and then the hemp oil/solvent combination is run through a filter press as a slurry. The solvent is then distilled off, resulting in pure hemp oil. The oil can then be further distilled and purified, if desired.

Extraction methods are designed to isolate compounds and, as such, will create concentrated products. In the case of hemp extraction, flower or chaff with ≤0.3% THC can be concentrated to levels ≥0.3%, thus disqualifying the extract as hemp.

Importantly the very low to zero THC in the varieties of the invention can avoid additional steps needed to separate or extract THC from resultant purified cannabinoids to bring it to levels ≤0.3%, such as by reverse-phased flash chromatography, a step often used to create an extract which can be legally defined as hemp.

In embodiments of the invention, the planting rate is greater than 14 pounds per acre and in further embodiments is greater than 15 pounds per acre, greater than 20 pounds per acre, greater than 25 pounds per acre, greater than 30 pounds per acre, greater than 35 pounds per acre or more or amounts in-between.

### Cannabinoid Preservation

Trichomes are epidermal appendages of diverse form, structure and function. They capture enough morphological variation to have been used in plant classification. Cannabis trichomes are rich in both cannabinoids and terpenes. However, the trichomes are fragile and readily damaged, which results in lower cannabinoid and terpene content of harvested product and limits growth of Cannabis for large scale commercial production of cannabinoids and terpenes. Cannabis has been traditionally grown in greenhouses and bred only for increased desired compounds such as THC and CBD. While improvements have been made in yields of these components, the varieties are only able to grow well in a tightly controlled environment. Furthermore, growers have to handle the Cannabis plants very carefully to avoid damaging the delicate trichomes.

The Cannabis cultivars of the invention provide increased cannabinoid preservation, particularly preservation during harvest. In some embodiments, the increased cannabinoid preservation is due to trichomes with increased integrity, thereby providing resistance to sources of trichome damage, including those associated with outdoor commercial production such as physical contact or agitation, high temperature, and high light. In some embodiments, the increased cannabinoid preservation is due to the plant architecture including, but not limited to, leaf positioning that provides protection of trichomes during harvest. Preferably, at least 60%, at least 70%, at least 80%, at least 90%, at least 95%, at least 98%, or at least 99% of the cannabinoids are preserved from breakage or damage. In some embodiments, the increased cannabinoid preservation is due to a higher trichome density. Preferably, at least 50%, at least 40%, at least 30%, at least 20%, at least 10%, at least 5%, at least 3%, at least 2%, or at least 1% of the trichome density is increased. In some embodiments, the increased cannabinoid content is due to specific morphological characteristics including, but not limited to, the mode of trichome ramification (e.g. simple, branched, few-armed, dendroid), unicellular or multicellular trichomes, the number and type of stalk cells, the number and type of gland cells, the cell wall composition of stalk cells, the cell wall composition of gland cells, the properties of the cuticular oil sac membrane, the size of the trichomes, the orientation of the trichomes (e.g. erect, sub-appressed or appressed) and the type of trichome surface (papillate or smooth). In some embodiments, the increased cannabinoid preservation is due to intracellular sequestration of the cannabinoids including, but not limited to, differences in subceullar location, concentration, or aggregation.

The fragility of the trichomes also previously limited the use of mechanized harvesting necessary for cost-effective large-scale commercial production. The increased preservation of the cannabinoids also enables traditional means of commercial production such as higher planting densities, resulting in higher yields per acre and a concomitant increase in the production of cannabinoids. In embodiments of the invention, the planting rate is greater than 14 pounds per acre and in further embodiments is greater than 15 pounds per acre, greater than 20 pounds per acre, greater than 25 pounds per acre, greater than 30 pounds per acre, greater than 35 pounds per acre or more or amounts in-between.

Trichome preservation can be assessed through a variety of approaches. For example, cannabinoids can be extracted from Cannabis plant tissue following harvest by hand and by machine. By comparing the quantity of cannabinoids obtained by a gentle approach (e.g. by hand) to a more aggressive harvesting approach (e.g. by machine), the relative loss of cannabinoids due to damage of the trichomes from machine harvesting can be determined. Preferably, the quantity of cannabinoids obtained from the machine harvest decreases by less than 40%, less than 30%, less than 25%, less than 20%, less than 15%, less than 10%, less than 5%, or less than 1% relative to harvesting by hand. Alternatively, the trichomes can be viewed under a microscope following harvest in order to assess damage to the trichomes and identify the type(s) of trichome morphology which are more resilient to mechanical harvest. Trichomes can similarly be assessed after subjecting the plant or plant part to other sources of damage such as high temperature, light, or oxygen.

### Cultivars of the Invention

Cannabis NWG331

Cannabis NWG331 is a hemp Cannabis cultivar with less than 0.2% of Δ9-Tetrahydrocannabinal (THC). The plants exhibit elevated levels of cannabidiol (CBD) and a ratio of CBD/THC of up to about 83:1. The cultivar produces plants with an average cannabidiol (CBD) content of more than 1.07% based upon total dry weight of the plant. It was generated from pedigree breeding with bulk and single seed descent selections methods and is genetically uniform and stable.

Cannabis NWG331 is a dioecious cultivar with male and female flowers that flowers at 58 days after planting. The plant height averages 170 cm to 190 cm and is medium height for a Cannabis cultivar. The plant has medium branching. The middle third of the plant is characterized by medium stem internode length, green stem color, green leaf color, medium leaf intensity, and medium leaf size. The cultivar has medium depth and width of stem grooves. Leaf anthocyanin coloration and male flower anthocyanin collation is absent. Hairs on the calyx are present but not in high density or length. Seed size is a thousand kernel weight of 13.5 grams and seed shape is spherical.

Table 1 below shows a typical profile of terpene content (ppm) for NWG331 as determined by head-space Gas Chromatography (Hs-GC) with flame ionization in female bud tissue.

TABLE 1

| | |
|---|---|
| a-pinene | 499.46 |
| Camphene | 7.55 |
| Sabinene | 6.87 |
| Myrcene | 209.55 |
| b-pinene | 120.12 |
| a-phellandrene | 12.43 |
| 3-carene | 11.19 |
| a-terpenine | 11.74 |
| Cineole | |
| ocimene-1 | 7.11 |
| Limonene | 9.42 |
| p-cymene | |
| ocimene-2 | 446.16 |
| Eucalyptol | 17.38 |
| g-terpenine | 10.00 |
| Terpinolene | 99.04 |
| Linalool | 19.01 |
| Fenchone | 4.88 |
| Isopulegol | 13.40 |
| Borneol | 4.78 |
| Terpineol | 18.83 |
| Citronellol | 6.49 |
| Geraniol | |
| citral-1 | |
| Pulegone | |
| citral-2 | 8.20 |
| b-caryophyllene | 1163.76 |
| Humulene | 309.15 |
| nerolidol-1 | 38.20 |
| nerolidol-2 | 29.75 |
| Guaiol | 10.39 |
| caryophyllene oxide | 19.81 |
| a-bisabolol | 65.61 |

Table 2 shows a typical cannabinoid content estimate as determined by High-performance liquid chromatography (% dry wt) in female bud tissue harvested from NWG331.

TABLE 2

| THC | THC- | THC- | CBD | CBD- | CBD- | CBN | CBG | |
|---|---|---|---|---|---|---|---|---|
| % | A % | total | % | A % | total | % | % | CBD:THC |
| 0.01 | 0.09 | 0.09 | 0.03 | 2.50 | 2.54 | 0.00 | 0.00 | 28.32 |

Cannabis NWG452

Cannabis NWG452 is a hemp Cannabis cultivar with less than 0.2% of Δ9-Tetrahydrocannabinal (THC). The plants exhibit elevated levels cannabidiol (CBD) and a ratio of CBD/THC of up to about 83:1. The cultivar produces plants with an average cannabidiol (CBD) content of more than 1.07% based upon total dry weight of the plant. It was generated from pedigree breeding with bulk and single-seed descent selections methods and is genetically uniform and stable.

Table 3 below shows a typical profile of terpene content (ppm) for NWG452 as determined by head-space Gas Chromatography (Hs-GC) with flame ionization in female bud tissue.

TABLE 3

| | |
|---|---|
| a-pinene | 269.10 |
| camphene | 6.28 |
| sabinene | 3.83 |
| myrcene | 311.61 |
| b-pinene | 81.61 |
| a-phellandrene | 10.58 |
| 3-carene | 6.24 |
| a-terpenine | 7.84 |
| cineole | |
| ocimene-1 | 12.72 |
| limonene | 11.03 |
| p-cymene | |
| ocimene-2 | 281.22 |
| eucalyptol | 26.07 |
| g-terpenine | 5.86 |
| terpinolene | 48.13 |
| linalool | 12.63 |
| fenchone | 4.34 |
| isopulegol | |
| borneol | 10.72 |
| terpineol | 15.79 |
| citronellol | 5.91 |
| geraniol | |
| citral-1 | |
| pulegone | 2.04 |
| citral-2 | 8.67 |
| b-caryophyllene | 1079.27 |
| humulene | 300.55 |
| nerolidol-1 | 19.55 |
| nerolidol-2 | 62.85 |
| guaiol | 11.23 |
| caryophyllene oxide | 23.46 |
| a-bisabolol | 30.89 |

The terpene profile of NWG452, shows that its myrcene content is 50% higher relative to NWG331.

Table 4 shows a typical cannabinoid content estimate as determined by High-performance liquid chromatography (% dry wt) in female bud tissue harvested from NWG452

TABLE 4

| THC | THC- | THC- | CBD | CBD- | CBD- | CBN | CBG | |
|---|---|---|---|---|---|---|---|---|
| % | A % | total | % | A % | total | % | % | CBD:THC |
| 0.00 | 0.10 | 0.10 | 0.01 | 2.82 | 2.83 | 0.00 | 0.00 | 28.0 |

### Further Embodiments of the Invention

This invention is also directed to methods for producing a Cannabis plant by crossing a first parent Cannabis plant with a second parent Cannabis plant, wherein the first parent Cannabis plant or second parent Cannabis plant is the Cannabis plant from cultivar NWG331 or NWG452. Further, both the first parent Cannabis plant and second parent Cannabis plant may be from cultivar NWG331 or NWG452. Therefore, any methods using hemp Cannabis cultivars NWG331 or NWG452 are part of this invention, such as selfing, backcrosses, hybrid breeding, and crosses to populations. Plants produced using hemp Cannabis cultivars of the invention as at least one parent are within the scope of this invention.

In one aspect of the invention, methods for developing novel plant types are presented. In one embodiment the specific type of breeding method is pedigree selection, where both single plant selection and mass selection practices are employed. Pedigree selection, also known as the “Vilmorin system of selection,” is described in Fehr, Walter; Principles of Cultivar Development, Volume I, Macmillan Publishing Co., which is hereby incorporated by reference.

In one embodiment, the pedigree method of breeding is practiced where selection is first practiced among F₂ plants. In the next season, the most desirable F₃ lines are first identified, and then desirable F₃ plants within each line are selected. The following season and in all subsequent generations of inbreeding, the most desirable families are identified first, then desirable lines within the selected families are chosen, and finally desirable plants within selected lines are harvested individually. A family refers to lines that were derived from plants selected from the same progeny row the preceding generation.

Using this pedigree method, two parents may be crossed using an emasculated female and a pollen donor (male) to produce F₁ offspring. The F₁ may be self-pollinated to produce a segregating F2 generation. Individual plants may then be selected which represent the desired phenotype in each generation (F3, F4, F5, etc.) until the traits are homozygous or fixed within a breeding population.

In addition to crossing, selection may be used to identify and isolate new Cannabis lines. In Cannabis selection, Cannabis seeds are planted, the plants are grown, and single plant selections are made of plants with desired characteristics. Seed from the single plant selections may be harvested, separated from seeds of the other plants in the field and re-planted. The plants from the selected seed may be monitored to determine if they exhibit the desired characteristics of the originally selected line. Selection work is preferably continued over multiple generations to increase the uniformity of the new line.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program may include a periodic, objective evaluation of the efficiency of the breeding procedure. Evaluation criteria vary depending on the goal and objectives but should include gain from selection per year based on comparisons to an appropriate standard, the overall value of the advanced breeding lines, and the number of successful cultivars produced per unit of input (e.g., per year, per dollar expended, etc.).

In one embodiment, promising advanced breeding lines are thoroughly tested and compared to appropriate standards in environments representative of the commercial target area(s). The best lines are candidates for new commercial cultivars; those still deficient in a few traits are used as parents to produce new populations for further selection.

These processes, which lead to the final step of marketing and distribution, usually take several years from the time the first cross or selection is made. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of Cannabis plant breeding is to develop new, unique and superior Cannabis cultivars. In one embodiment, the breeder initially selects and crosses two or more parental lines, followed by repeated selfing and selection, producing many new genetic combinations. The breeder can theoretically generate billions of different genetic combinations via crossing, selfing and mutations. Preferably, each year the plant breeder selects the germplasm to advance to the next generation. This germplasm may be grown under different geographical, climatic and soil conditions, and further selections are then made, during and at the end of the growing season.

In a preferred embodiment, the development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Pedigree breeding is used commonly for the improvement of self-pollinating crops or inbred lines of cross-pollinating crops. Two parents which possess favorable, complementary traits are crossed to produce an F₁. An F2 population is produced by selfing one or several F₁'s or by intercrossing two F₁'s (sib mating). Selection of the best individuals is usually begun in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

The single-seed descent procedure refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the identification of markers which are closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Descriptions of other breeding methods that are commonly used for different traits and crops can be found in one of several reference books (e.g., Principles of Plant Breeding John Wiley and Son, pp. 115-161, 1960; Allard, 1960; Simmonds, 1979; Sneep et al., 1979; Fehr, 1987; “Carrots and Related Vegetable Umbelliferae”, Rubatzky, V. E., et al., 1999).

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars.

This invention also is directed to methods for producing a Cannabis cultivar plant by crossing a first parent Cannabis plant with a second parent Cannabis plant wherein either the first or second parent Cannabis plant is a Cannabis plant of the line NWG331 or NWG452. Further, both first and second parent Cannabis plants can come from the cultivar NWG331 or NWG452. Still further, this invention also is directed to methods for producing a cultivar NWG331 or NWG452-derived Cannabis plant by crossing cultivar NWG331 or NWG452 with a second Cannabis plant and growing the progeny seed and repeating the crossing and growing steps with the cultivar NWG331 or NWG452-derived plant from 0 to 7 times. Thus, any such methods using the cultivar NWG331 or NWG452 are part of this invention: selfing, backcrosses, hybrid production, crosses to populations, and the like. All plants produced using cultivar NWG331 or NWG452 as a parent are within the scope of this invention, including plants derived from cultivar NWG331 or NWG452. Advantageously, the cultivar is used in crosses with other, different, cultivars to produce first generation (F₁) Cannabis seeds and plants with superior characteristics.

As used herein, the term plant includes plant cells, plant protoplasts, plant cell tissue cultures from which Cannabis plants can be regenerated, plant calli, plant clumps and plant cells that are intact in plants or parts of plants, such as embryos, pollen, ovules, flowers, seeds, roots, anthers, and the like.

As is well known in the art, tissue culture of Cannabis can be used for the in vitro regeneration of a Cannabis plant. Tissue culture of various tissues of Cannabis and regeneration of plants therefrom is well known and widely published. For example, reference may be had to Teng et al., HortScience. 1992, 27: 9, 1030-1032 Teng et al., HortScience. 1993, 28: 6, 669-1671, Zhang et al., Journal of Genetics and Breeding. 1992, 46: 3, 287-290, Webb et al., Plant Cell Tissue and Organ Culture. 1994, 38: 1, 77-79, Curtis et al., Journal of Experimental Botany. 1994, 45: 279, 1441-1449, Nagata et al., Journal for the American Society for Horticultural Science. 2000, 125: 6, 669-672. It is clear from the literature that the state of the art is such that these methods of obtaining plants are, and were, “conventional” in the sense that they are routinely used and have a very high rate of success. Thus, another aspect of this invention is to provide cells which upon growth and differentiation produce Cannabis plants having the physiological and morphological characteristics of variety NWG331 or NWG452.

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as transgenes. Over the last fifteen to twenty years several methods for producing transgenic plants have been developed, and the present invention, in particular embodiments, also relates to transformed versions of the claimed line.

Plant transformation preferably involves the construction of an expression vector that will function in plant cells. Such a vector may comprise DNA comprising a gene under control of or operatively linked to a regulatory element (for example, a promoter). The expression vector may contain one or more such operably linked gene/regulatory element combinations. The vector(s) may be in the form of a plasmid and can be used alone or in combination with other plasmids, to provide transformed Cannabis plants, using transformation methods as described below to incorporate transgenes into the genetic material of the Cannabis plant(s).

### Expression Vectors for Cannabis Transformation

### Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or a herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which when placed under the control of plant regulatory signals confers resistance to kanamycin. Fraley et al., Proc. Natl. Acad. Sci. U.S.A., 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase, aminoglycoside-3′-adenyl transferase, the bleomycin resistance determinant. Hayford et al., Plant Physiol. 86:1216 (1988), Jones et al., Mol. Gen. Genet., 210:86 (1987), Svab et al., Plant Mol. Biol. 14:197 (1990<Hille et al., Plant Mol. Biol. 7:171 (1986). Other selectable marker genes confer resistance to herbicides such as glyphosate, glufosinate or broxynil. Comai et al., Nature 317:741-744 (1985), Gordon-Kamm et al., Plant Cell 2:603-618 (1990) and Stalker et al., Science 242:419-423 (1988).

Other selectable marker genes for plant transformation are not of bacterial origin. These genes include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker gene for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include beta-glucuronidase (GUS), beta-galactosidase, luciferase and chloramphenicol, acetyltransferase. Jefferson, R. A., Plant Mol. Biol. Rep. 5:387 (1987), Teen et al., EMBO J. 8:343 (1989), Koncz et al., Proc. Natl. Acad. Sci U.S.A. 84:131 (1987), DeBlock et al., EMBO J. 3:1681 (1984).

Recently, in vivo methods for visualizing GUS activity that do not require destruction of plant tissue have been made available. Molecular Probes publication 2908, Imagene Green™, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Promoters

Genes included in expression vectors preferably are driven by nucleotide sequence comprising a regulatory element, for example, a promoter. Several types of promoters are now well known in the transformation arts, as are other regulatory elements that can be used alone or in combination with promoters.

As used herein, promoter includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissue are referred to as “tissue-specific”. A “cell type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive promoter” is a promoter which is active under most environmental conditions.

A. Inducible Promoters

An inducible promoter is operably linked to a gene for expression in Cannabis. Optionally, the inducible promoter is operably linked to a nucleotide sequence encoding a signal sequence which is operably linked to a gene for expression in Cannabis. With an inducible promoter the rate of transcription increases in response to an inducing agent. Any inducible promoter can be used in the instant invention. See Ward et al., Plant Mol. Biol. 22:361-366 (1993). Exemplary inducible promoters include, but are not limited to, that from the ACEI system which responds to copper (Meft et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. U.S.A. 88:0421 (1991).

B. Constitutive Promoters

A constitutive promoter may be operably linked to a gene for expression in Cannabis or the constitutive promoter may operably link to a nucleotide sequence encoding a signal sequence which is operably linked to a gene for expression in Cannabis.

Many different constitutive promoters can be utilized in the instant invention. Exemplary constitutive promoters include, but are not limited to, the promoters from plant viruses such as the 35S promoter from CaMV (Odell et al., Nature 313:810-812 (1985) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO96/30530.

C. Tissue-Specific or Tissue-Preferred Promoters

A tissue-specific promoter may be operably linked to a gene for expression in Cannabis. Optionally, the tissue-specific promoter is operably linked to a nucleotide sequence encoding a signal sequence which is operably linked to a gene for expression in Cannabis. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993).

### Signal Sequences for Targeting Proteins to Subcellular Compartments

Transport of protein produced by transgenes to a subcellular compartment such as the chloroplast, vacuole, peroxisome, glyoxysome, cell wall or mitochondroin or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example Becker et al., Plant Mol. Biol. 20:49 (1992), Close, P. S., Master's Thesis, Iowa State University (1993), Knox, C., et al., Structure and Organization of Two Divergent Alpha-Amylase Genes from Barley, Plant Mol. Biol. 9:3-17 (1987), Lerner et al., Plant Physiol. 91:124-129 (1989), Fontes et al., Plant Cell 3:483-496 (1991), Matsuoka et al., Proc. Natl. Acad. Sci. 88:834 (1991), Gould et al., J. Cell. Biol. 108:1657 (1989), Creissen et al., Plant J. 2:129 (1991), Kalderon, et al., A short amino acid sequence able to specify nuclear location, Cell 39:499-509 (1984), Steifel, et al., Expression of a maize cell wall hydroxyproline-rich glycoprotein gene in early leaf and root vascular differentiation, Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants that are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is Cannabis. In another preferred embodiment, the biomass of interest is seed. For transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons may involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes that Confer Resistance to Pests or Disease and that Encode:

A. Plant disease resistance genes. Plant defenses are often activated by specific interaction between the product of a disease resistance gene (R) in the plant and the product of a corresponding avirulence (Avr) gene in the pathogen. A plant line can be transformed with cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. Tomato encodes a protein kinase); Mindrinos et al., Cell 78:1089 (1994) (Arabidopsis RSP2 gene for resistance to Pseudomonas syringae).

B. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

C. A lectin. See, for example, the disclosure by Van Damme et al., Plant Molec. Biol. 24:25 (1994), who disclose the nucleotide sequences of several Clivia miniata mannose-binding lectin genes.

D. A vitamin-binding protein such as avidin. See PCT application US93/06487, the contents of which are hereby incorporated by reference. The application teaches the use of avidin and avidin homologues as larvicides against insect pests.

E. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotoch. Biochem. 57:1243 (1993) (nucleotide sequence of Streptomyces nitrosporeus α-amylase inhibitor).

F. An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

G. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

H. An insect-specific venom produced in nature by a snake, a wasp, etc. For example, see Pang et al., Gene 116:165 (1992), for disclosure of heterologous expression in plants of a gene coding for a scorpion insectotoxic peptide.

I. An enzyme responsible for a hyper accumulation of a monterpene, a sesquiterpene, a steroid, hydroxamic acid, a phenylpropanoid derivative or another non-protein molecule with insecticidal activity.

J. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hookworm chitinase, and Kawalleck et al., Plant Molec. Biol. 21:673 (1993), who provide the nucleotide sequence of the parsley ubi4-2 polyubiquitin gene.

K. A molecule that stimulates signal transduction. For example, see the disclosure by Botella et al., Plant Molec. Biol. 24:757 (1994), of nucleotide sequences for mung Cannabis calmodulin cDNA clones, and Griess et al., Plant Physiol. 104:1467 (1994), who provide the nucleotide sequence of a maize calmodulin cDNA clone.

L. A hydrophobic moment peptide. See PCT application WO95/16776 (disclosure of peptide derivatives of tachyolesin which inhibit fungal plant pathogens) and PCT application WO95/18855 (teaches synthetic antimicrobial peptides that confer disease resistance), the respective contents of which are hereby incorporated by reference.

M. A membrane permease, a channel former or a channel blocker. For example, see the disclosure of Jaynes et al., Plant Sci 89:43 (1993), of heterologous expression of a cecropin-β, lytic peptide analog to render transgenic tobacco plants resistant to Pseudomonas solanacearum.

N. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

O. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

P. A virus-specific antibody. See, for example, Tavladoraki et al., Nature 366:469 (1993), who show that transgenic plants expressing recombinant antibody genes are protected from virus attack.

Q. A developmental-arrestive protein produced in nature by a pathogen or a parasite. Thus, fungal endo-α-1, 4-D-polygalacturonases facilitate fungal colonization and plant nutrient release by solubilizing plant cell wall homo-α-1, 4-D-galacturonase. See Lamb at al., Bio/Technology 10:1436 (1992). The cloning and characterization of a gene which encodes a Cannabis endopolygalacturonase-inhibiting protein is described by Toubart et al., Plant J. 2:367 (1992).

R. A development-arrestive protein produced in nature by a plant. For example, Logemann et al., Bioi/Technology 10:305 (1992), have shown that transgenic plants expressing the barley ribosome-inactivating gene have an increased resistance to fungal disease.

S. A Cannabis mosaic potyvirus (LMV) coat protein gene introduced into Lactuca sativa in order to increase its resistance to LMV infection. See Dinant et al., Molecular Breeding. 1997, 3: 1, 75-86.

2. Genes that Confer Resistance to an Herbicide, for Example:

A. An herbicide that inhibits the growing point or meristem, such as an imidazalinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance impaired by mutant 5-enolpyruvl-3-phosphikimate synthase (EPSP) and aroA genes, respectively) and other phosphono compounds such as glufosinate (phosphinothricin acetyl transferase, PAT and Streptomyces hygroscopicus phosphinothricin-acetyl transferase PAT bar genes), and pyridinoxy or phenoxy propionic acids and cycloshexones (ACCase inhibitor-encoding genes). See, for example, U.S. Pat. No. 4,940,835 to Shah, et al., which discloses the nucleotide sequence of a form of EPSP which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. See also Umaballava-Mobapathie in Transgenic Research. 1999, 8: 1, 33-44 that discloses Lactuca sativa resistant to glufosinate. European patent application No. 0 333 033 to Kumada at al., and U.S. Pat. No. 4,975,374 to Goodman et al., disclose nucleotide sequences of glutamine synthetase genes which confer resistance to herbicides such as L-phosphinothricin. The nucleotide sequence of a phosphinothricin-acetyl-transferase gene is provided in European application No. 0 242 246 to Leemans et al., DeGreef et al., Bio/Technology 7:61 (1989), describe the production of transgenic plants that express chimeric bar genes coding for phosphinothricin acetyl transferase activity. Exemplary of genes conferring resistance to phenoxy propionic acids and cycloshexones, such as sethoxydim and haloxyfop are the Accl-S1, Accl-S2 and Accl-S3 genes described by Marshall et al., Theor. Appl. Genet. 83:435 (1992).

C. An herbicide that inhibits photosynthesis, such as a triazine (psbA and gs+ genes) and a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

D. Acetohydroxy acid synthase, which has been found to make plants that express this enzyme resistant to multiple types of herbicides, has been introduced into a variety of plants. See Hattori et al., Mol. Gen. Genet. 246:419, 1995. Other genes that confer tolerance to herbicides include a gene encoding a chimeric protein of rat cytochrome P4507A1 and yeast NADPH-cytochrome P450 oxidoreductase (Shiota et al., Plant Physiol., 106:17, 1994), genes for glutathione reductase and superoxide dismutase (Aono et al., Plant Cell Physiol. 36:1687, 1995), and genes for various phosphotransferases (Datta et al., Plant Mol. Biol. 20:619, 1992).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; 5,767,373; and international publication WO 01/12825.

3. Genes that Confer or Contribute to a Value-Added Trait, Such as:

A. Increased iron content of the Cannabis, for example by transforming a plant with a soybean ferritin gene as described in Goto et al., Acta Horticulturae. 2000, 521, 101-109. Parallel to the improved iron content enhanced growth of transgenic Cannabis s was also observed in early development stages.

B. Decreased nitrate content of leaves, for example by transforming a Cannabis with a gene coding for a nitrate reductase. See for example Curtis et al., Plant Cell Report. 1999, 18: 11, 889-896.

C. Increased sweetness of the Cannabis by transferring a gene coding for monellin that elicits a flavor sweeter than sugar on a molar basis. See Penarrubia et al., Biotechnology. 1992, 10: 5, 561-564.

D. Modified fatty acid metabolism, for example, by transforming a plant with an antisense gene of stearyl-ACP desaturase to increase stearic acid content of the plant. See Knultzon et al., Proc. Natl. Acad. Sci. USA 89:2625 (1992).

E. Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus licheniformis alpha-amylase), Elliot et al., Plant Molec. Biol. 21:515 (1993) (nucleotide sequences of tomato invertase genes), Sogaard et al., J. Biol. Chem. 268:22480 (1993) (site-directed mutagenesis of barley alpha amylase gene), and Fisher et al., Plant Physiol. 102:1045 (1993) (maize endosperm starch branching enzyme II).

4. Genes that Control Male-Sterility

A. Introduction of a deacetylase gene under the control of a tapetum-specific promoter and with the application of the chemical N-Ac-PPT. See international publication WO 01/29237.

B. Introduction of various stamen-specific promoters. See international publications WO 92/13956 and WO 92/13957.

C. Introduction of the barnase and the barstar genes. See Paul et al., Plant Mol. Biol. 19:611-622, 1992).

### Methods for Cannabis Transformation

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 89-119.

A. Agrobacterium-Mediated Transformation

One method for introducing an expression vector into plants is based on the natural transformation system of Agrobacterium. See, for example, Horsch et al., Science 227:1229 (1985). Curtis et al., Journal of Experimental Botany. 1994, 45: 279, 1441-1449, Torres et al., Plant cell Tissue and Organic Culture. 1993, 34: 3, 279-285, Dinant et al., Molecular Breeding. 1997, 3: 1, 75-86. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra, and Moloney et al., Plant Cell Reports 8:238 (1989). See also, U.S. Pat. No. 5,591,616 issued Jan. 7, 1997.

B. Direct Gene Transfer

Several methods of plant transformation collectively referred to as direct gene transfer have been developed as an alternative to Agrobacterium-mediated transformation. A generally applicable method of plant transformation is microprojectile-mediated transformation wherein DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Russell, D. R., et al. Pl. Cell. Rep. 12(3, January), 165-169 (1993), Aragao, F. J. L., et al. Plant Mol. Biol. 20(2, October), 357-359 (1992), Aragao, F. J. L., et al. Pl. Cell. Rep. 12(9, July), 483-490 (1993). Aragao, Theor. Appl. Genet. 93: 142-150 (1996), Kim, J.; Minamikawa, T. Plant Science 117: 131-138 (1996), Sanford et al., Part. Sci. Technol. 5:27 (1987), Sanford, J. C., Trends Biotech. 6:299 (1988), Klein et al., Bio/Technology 6:559-563 (1988), Sanford, J. C., Physiol Plant 7:206 (1990), Klein et al., Biotechnology 10:268 (1992).

Another method for physical delivery of DNA to plants is sonication of target cells. Zhang et al., Bio/Technology 9:996 (1991). Alternatively, liposome or spheroplast fusion has been used to introduce expression vectors into plants. Deshayes et al., EMBO J., 4:2731 (1985), Christou et al., Proc Natl. Acad. Sci. U.S.A. 84:3962 (1987). Direct uptake of DNA into protoplasts using CaCl₂ precipitation, polyvinyl alcohol or poly-L-omithine has also been reported. Hain et al., Mol. Gen. Genet. 199:161 (1985) and Draper et al., Plant Cell Physiol. 23:451 (1982). Electroporation of protoplasts and whole cells and tissues have also been described. Saker, M.; Kuhne, T. Biologia Plantarum 40(4): 507-514 (1997/98), Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994). See also Chupean et al., Biotechnology. 1989, 7: 5, 503-508.

Following transformation of Cannabis target tissues, expression of the above-described selectable marker genes allows for preferential selection of transformed cells, tissues and/or plants, using regeneration and selection methods now well known in the art.

The foregoing methods for transformation would typically be used for producing a transgenic line. The transgenic line could then be crossed, with another (non-transformed or transformed) line, in order to produce a new transgenic Cannabis line. Alternatively, a genetic trait that has been engineered into a particular Cannabis cultivar using the foregoing transformation techniques could be moved into another line using traditional backcrossing techniques that are well known in the plant breeding arts. For example, a backcrossing approach could be used to move an engineered trait from a public, non-elite inbred line into an elite inbred line, or from an inbred line containing a foreign gene in its genome into an inbred line or lines which do not contain that gene. As used herein, “crossing” can refer to a simple X by Y cross, or the process of backcrossing, depending on the context.

### Tilling

In one embodiment, TILLING (Targeting Induced Local Lesions IN Genomes) can be used to produce plants in which endogenous genes comprise a mutation, for example genes that increase trichome integrity. In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time. For a TILLING assay, heteroduplex methods using specific endonucleases can be used to detect single nucleotide polymorphisms (SNPs). Alternatively, Next Generation Sequencing of DNA from pools of mutagenised plants can be used to identify mutants in the gene of choice. Typically, a mutation frequency of one mutant per 1000 plants in the mutagenised population is achieved. Using this approach, many thousands of plants can be screened to identify any individual with a single base change as well as small insertions or deletions (1-30 bp) in any gene or specific region of the genome. TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

### Genome Editing Using Site-Specific Nucleases

Genome editing uses engineered nucleases such as RNA guided DNA endonucleases or nucleases composed of sequence specific DNA binding domains fused to a non-specific DNA cleavage module. These engineered nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption. Engineered nucleases useful in the methods of the present invention include zinc finger nucleases (ZFNs), transcription activator-like (TAL) effector nucleases (TALEN) and CRISPR/Cas9 type nucleases.

Typically, nuclease encoded genes are delivered into cells by plasmid DNA, viral vectors or in vitro transcribed mRNA. A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell or organism. Typically, a ZFN having more than three zinc fingers would have progressively greater specificity with each additional zinc finger.

The zinc finger domain can be derived from any class or type of zinc finger. In a particular embodiment, the zinc finger domain comprises the Cis2His2 type of zinc finger that is very generally represented, for example, by the zinc finger transcription factors TFIIIA or Spl. In a preferred embodiment, the zinc finger domain comprises three Cis2His2 type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques. (see, for example, Bibikova et al., 2002).

The ZFN DNA-cleavage domain is derived from a class of non-specific DNA cleavage domains, for example the DNA-cleavage domain of a Type II restriction enzyme such as Fold (Kim et al., 1996). Other useful endonucleases may include, for example, HhalI, HindIII, Nod, BbvCI, EcoRI, BglI, and AlwI.

A transcription activator-like (TAL) effector nuclease (TALEN) comprises a TAL effector DNA binding domain and an endonuclease domain. TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AhvI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

### Genome Editing Using Programmable RNA-Guided DNA Endonucleases

Distinct from the site-specific nucleases described above, the clustered regulatory interspaced short palindromic repeats (CRISPR)/Cas system provides an alternative to ZFNs and TALENs for inducing targeted genetic alterations, via RNA-guided DNA cleavage.

CRISPR systems rely on CRISPR RNA (crRNA) and transactivating chimeric RNA (tracrRNA) for sequence-specific cleavage of DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

The CRISPR system can be portable to plant cells by co-delivery of plasmids expressing the Cas endonuclease and the necessary crRNA components. The Cas endonuclease may be converted into a nickase to provide additional control over the mechanism of DNA repair (Cong et al., 2013).

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

### Gene Conversions

When the term Cannabis plant, cultivar or Cannabis line is used in the context of the present invention, this also includes any gene conversions of that line. The term gene converted plant as used herein refers to those Cannabis plants which are developed by a plant breeding technique called backcrossing wherein essentially all of the desired morphological and physiological characteristics of a cultivar are recovered in addition to the gene transferred into the line via the backcrossing technique. Backcrossing methods can be used with the present invention to improve or introduce a characteristic into the line. The term backcrossing as used herein refers to the repeated crossing of a hybrid progeny back to one of the parental Cannabis plants for that line. The parental Cannabis plant that contributes the gene for the desired characteristic is termed the nonrecurrent or donor parent. This terminology refers to the fact that the nonrecurrent parent is used one time in the backcross protocol and therefore does not recur. The parental Cannabis plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original cultivar of interest (recurrent parent) is crossed to a second line (nonrecurrent parent) that carries the single gene of interest to be transferred. The resulting progeny from this cross are then crossed again to the recurrent parent and the process is repeated until a Cannabis plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute traits or characteristics in the original line. To accomplish this, a gene or genes of the recurrent cultivar are modified or substituted with the desired gene or genes from the nonrecurrent parent, while retaining essentially all of the rest of the desired genetic, and therefore the desired physiological and morphological, constitution of the original line. The choice of the particular nonrecurrent parent will depend on the purpose of the backcross. One of the major purposes is to add some commercially desirable, agronomically important trait or traits to the plant. The exact backcrossing protocol will depend on the characteristics or traits being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many gene traits have been identified that are not regularly selected for in the development of a new line but that can be improved by backcrossing techniques. Gene traits may or may not be transgenic, examples of these traits include but are not limited to, herbicide resistance, resistance for bacterial, fungal, or viral disease, insect resistance, enhanced nutritional quality, industrial usage, yield stability, yield enhancement, male sterility, modified fatty acid metabolism, and modified carbohydrate metabolism. These genes are generally inherited through the nucleus. Several of these gene traits are described in U.S. Pat. Nos. 5,777,196; 5,948,957 and 5,969,212, the disclosures of which are specifically hereby incorporated by reference.

### Tissue Culture

Further reproduction of the variety can occur by tissue culture and regeneration. Tissue culture of various tissues of Cannabis and regeneration of plants therefrom is well known and widely published. For example, reference may be had to Teng et al., HortScience. 1992, 27: 9, 1030-1032 Teng et al., HortScience. 1993, 28: 6, 669-1671, Zhang et al., Journal of Genetics and Breeding. 1992, 46: 3, 287-290, Webb et al., Plant Cell Tissue and Organ Culture. 1994, 38: 1, 77-79, Curtis et al., Journal of Experimental Botany. 1994, 45: 279, 1441-1449, Nagata et al., Journal for the American Society for Horticultural Science. 2000, 125: 6, 669-672, and Ibrahim et al., Plant Cell, Tissue and Organ Culture. (1992), 28(2): 139-145. It is clear from the literature that the state of the art is such that these methods of obtaining plants are routinely used and have a very high rate of success. Thus, another aspect of this invention is to provide cells which upon growth and differentiation produce Cannabis plants having the physiological and morphological characteristics of cultivar NWG331 or NWG452.

As used herein, the term “tissue culture” indicates a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, suckers and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

### Additional Breeding Methods

This invention also is directed to methods for producing a Cannabis plant by crossing a first parent Cannabis plant with a second parent Cannabis plant wherein the first or second parent Cannabis plant is a Cannabis plant of cultivar NWG331 or NWG452. Further, both first and second parent Cannabis plants can come from hemp Cannabis cultivars of the invention. Thus, any such methods using hemp Cannabis cultivars of the invention are part of this invention: selfing, backcrosses, hybrid production, crosses to populations, and the like. All plants produced using hemp Cannabis cultivars of the invention as at least one parent is within the scope of this invention, including those developed from cultivars derived from hemp Cannabis cultivars of the invention. Advantageously, this Cannabis cultivar could be used in crosses with other, different, Cannabis plants to produce the first generation (F₁) Cannabis hybrid seeds and plants with superior characteristics. The cultivar of the invention can also be used for transformation where exogenous genes are introduced and expressed by the cultivar of the invention. Genetic variants created either through traditional breeding methods using hemp Cannabis cultivars of the invention or through transformation of cultivar NWG331 or NWG452 by any of a number of protocols known to those of skill in the art are intended to be within the scope of this invention.

The following describes breeding methods that may be used with hemp Cannabis cultivars of the invention in the development of further Cannabis plants. One such embodiment is a method for developing cultivar NWG331 or NWG452 progeny Cannabis plants in a Cannabis plant breeding program comprising: obtaining the Cannabis plant, or a part thereof, of cultivar NWG331 or NWG452, utilizing said plant or plant part as a source of breeding material, and selecting a hemp Cannabis cultivars of the invention progeny plant with molecular markers in common with cultivar NWG331 or NWG452 and/or with morphological and/or physiological characteristics selected from the characteristics listed in Table 1. Breeding steps that may be used in the Cannabis plant breeding program include pedigree breeding, backcrossing, mutation breeding, and recurrent selection. In conjunction with these steps, techniques such as RFLP-enhanced selection, genetic marker enhanced selection (for example SSR markers) and the making of double haploids may be utilized.

Another method which may be used involves producing a population of hemp Cannabis cultivars of the invention-progeny Cannabis plants, comprising crossing cultivar NWG331 or NWG452 with another Cannabis plant, thereby producing a population of Cannabis plants, which, on average, derive 50% of their alleles from hemp Cannabis cultivars of the invention. A plant of this population may be selected and repeatedly selfed or sibbed with a Cannabis cultivar resulting from these successive filial generations. One embodiment of this invention is the Cannabis cultivar produced by this method and that has obtained at least 50% of its alleles from hemp Cannabis cultivars of the invention.

One of ordinary skill in the art of plant breeding would know how to evaluate the traits of two plant varieties to determine if there is no significant difference between the two traits expressed by those varieties. For example, see Fehr and Walt, Principles of Cultivar Development, p 261-286 (1987). Thus the invention includes hemp Cannabis cultivars of the invention progeny Cannabis plants comprising a combination of at least two cultivar NWG331 or NWG452 traits selected from the group consisting of those listed in Table 1 or the cultivar NWG331 or NWG452 combination of traits listed above, so that said progeny Cannabis plant is not significantly different for said traits than hemp Cannabis cultivars of the invention as determined at the 5% significance level when grown in the same environmental conditions. Using techniques described herein, molecular markers may be used to identify said progeny plant as a hemp Cannabis cultivar of the invention progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of hemp Cannabis cultivars of the invention may also be characterized through their filial relationship with hemp Cannabis cultivars of the invention, as for example, being within a certain number of breeding crosses of hemp Cannabis cultivars of the invention. A breeding cross is a cross made to introduce new genetics into the progeny, and is distinguished from a cross, such as a self or a sib cross, made to select among existing genetic alleles. The lower the number of breeding crosses in the pedigree, the closer the relationship between hemp Cannabis cultivars of the invention and its progeny. For example, progeny produced by the methods described herein may be within 1, 2, 3, 4 or 5 breeding crosses of hemp Cannabis cultivars of the invention.

The foregoing invention has been described in detail by way of illustration and example for purposes of clarity and understanding. However, it will be obvious that certain changes and modifications such as single gene modifications and mutations, somoclonal variants, variant individuals selected from large populations of the plants of the instant variety and the like may be practiced within the scope of the invention, as limited only by the scope of the appended claims.

### EXAMPLES

### Example 1: Analysis of Trichome Response to Threshing

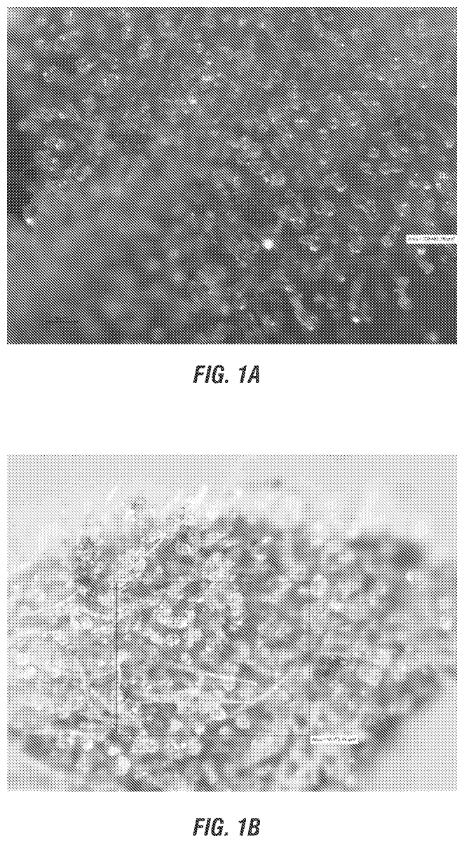

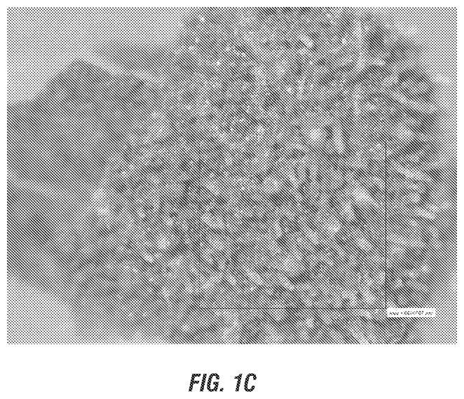

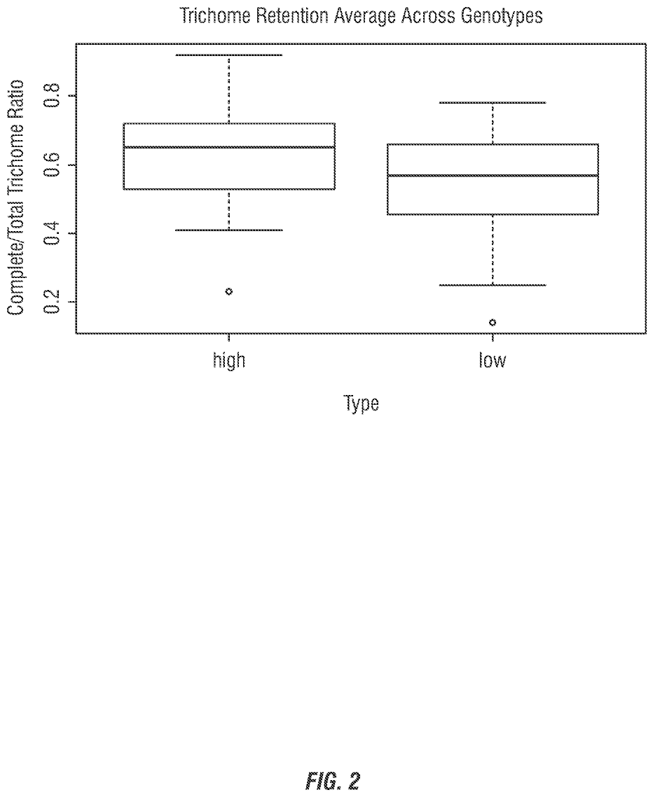

Trichome response to threshing was analyzed across four high CBD genotypes (9.7%) and four low CBD (1.7%) genotypes. Images were scored for intact and broken trichomes post-threshing (FIGS. 1A-C), and the ratio of intact trichomes to total trichomes was calculated. A significant difference (2-sample t-test, 1 tailed, N=5 per genotype) in mean trichome retention was observed (Table 5, FIG. 2). Genotypes with higher CBD content post-threshing retain more trichomes than those with lower CBD content.

TABLE 5

| | Mean | | | | |
|---|---|---|---|---|---|
| | Trichome | | | | |
| Type | Retention | Sd | test stat | df | p-value |
| High CBD | 0.64 | 0.16 | 1.94 | 38 | 0.0297 |
| Low CBD | 0.53 | 0.19 | | | |

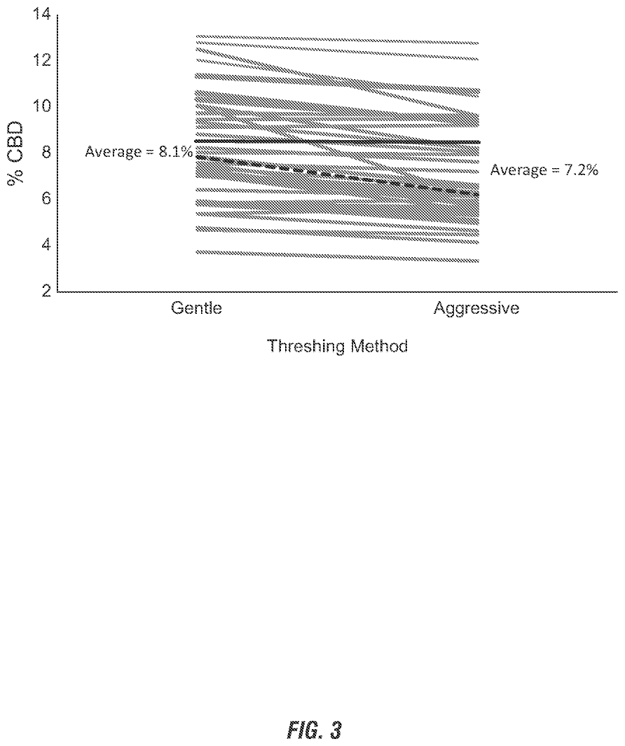

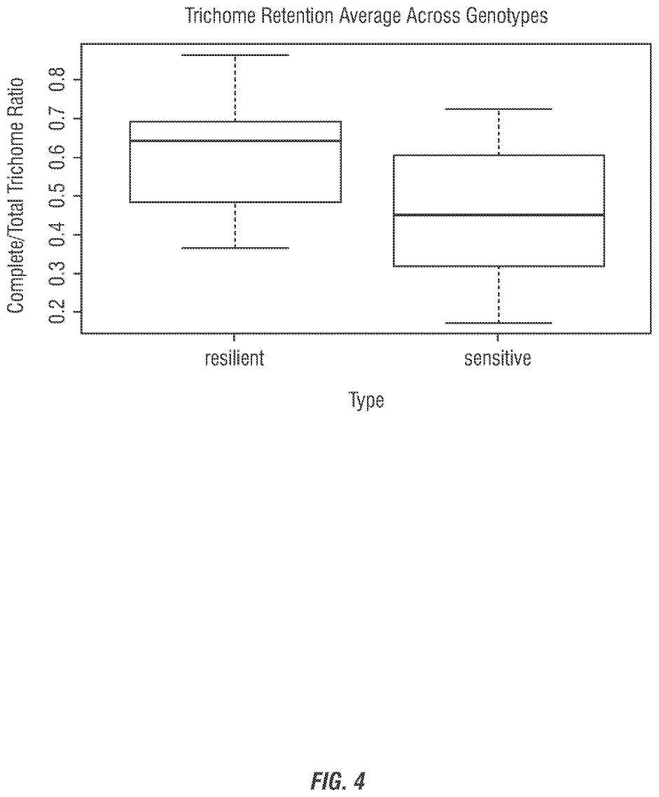

Genotypes with high cannabinoid preservation were then identified by comparing aggressive and gentle threshing methods. A total of 43 genotypes were hand threshed (gentle) and aggressively threshed to identify those with high retention or loss of cannabinoids (FIG. 3). Two genotypes (susceptible 2833 and resilient 2909) were analyzed in more detail. A significant difference in trichome retention was observed for replicate field samples of aggressively threshed 2909 and 2833 (Table 6, FIG. 4).

TABLE 6

| | Mean | | | | |
|---|---|---|---|---|---|
| | Trichome | | | | |
| Type | Retention | Sd | test stat | df | p-value |
| Resilient | 0.6 | 0.14 | 3.29 | 48 | >0.001 |
| (2909) | | | | | |
| Sensitive | 0.46 | 0.16 | | | |
| (2833) | | | | | |

### Example 2: Methods of Cannabis Production

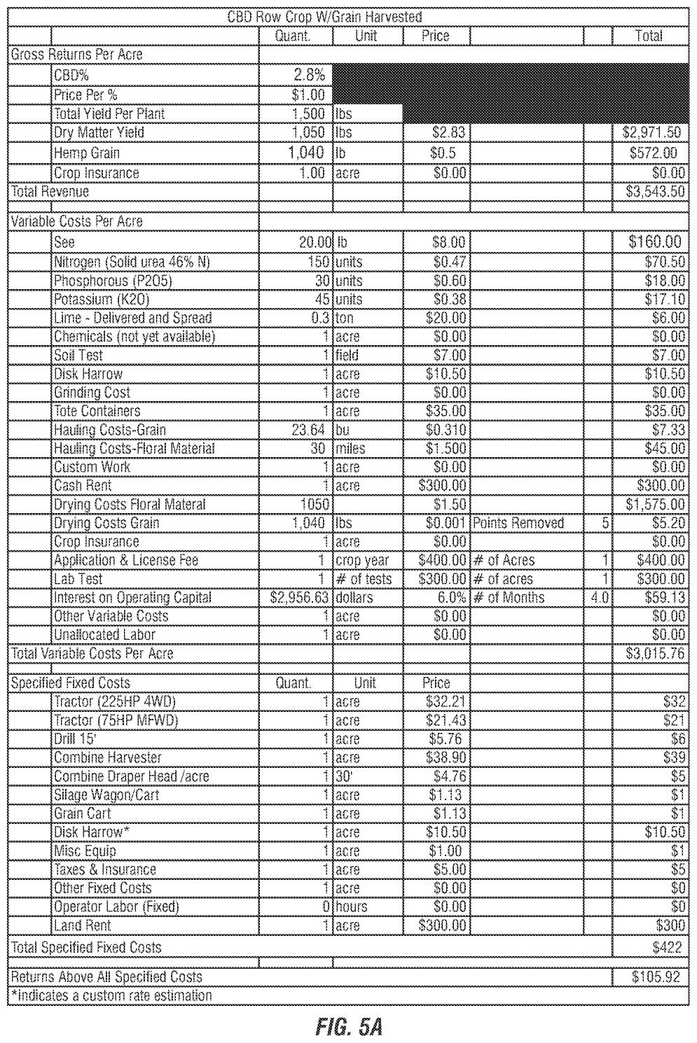

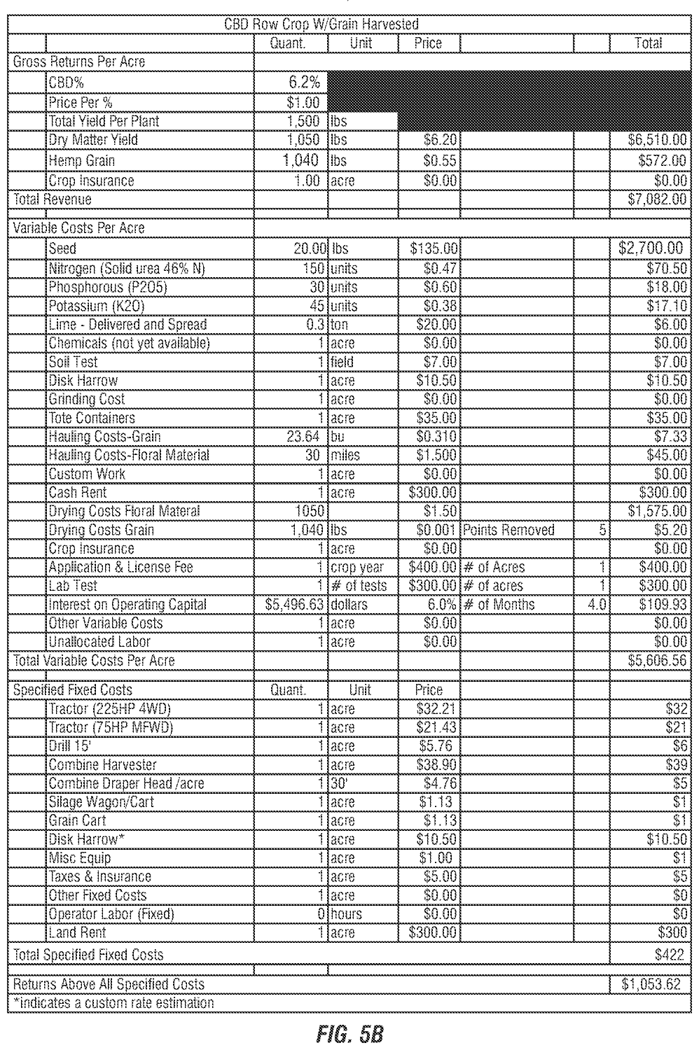

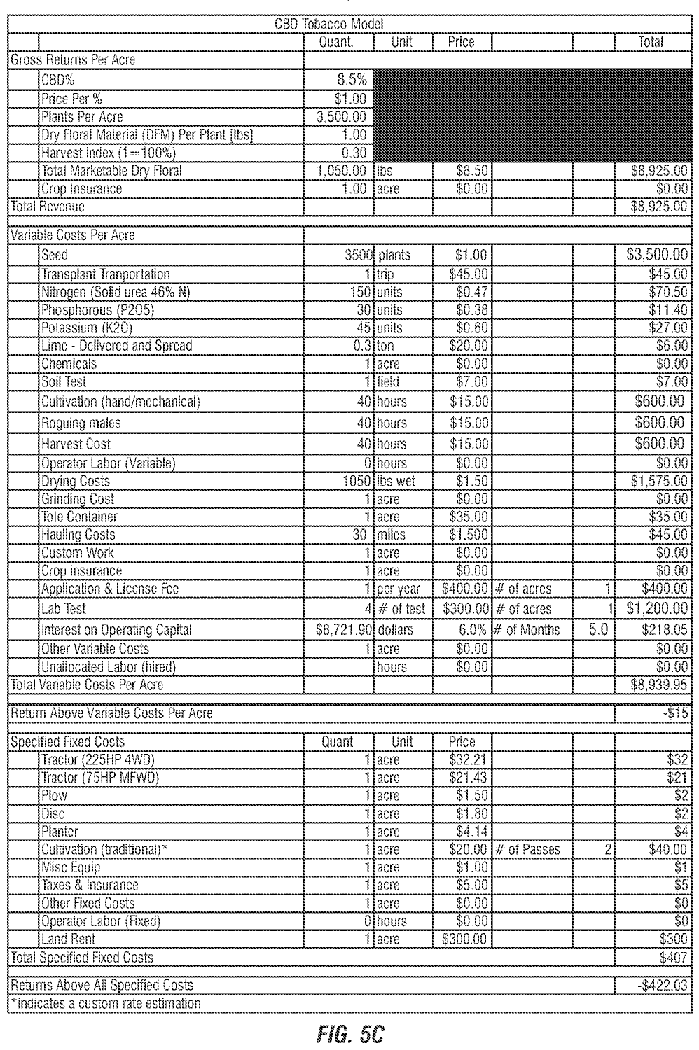

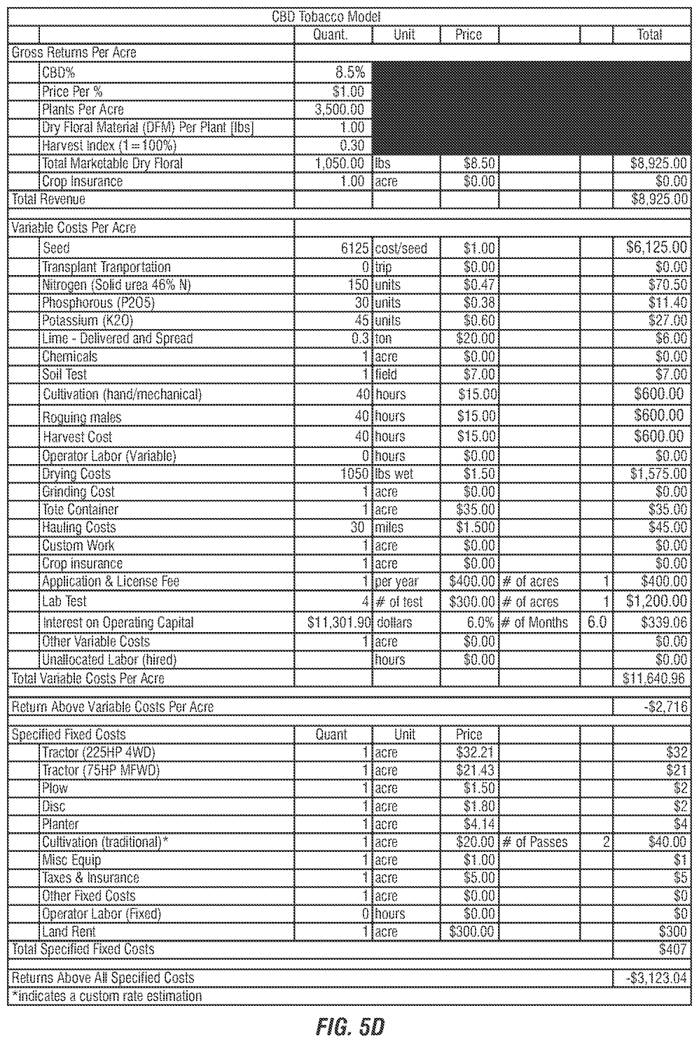

Three methods of production have been compared for costs and estimates of overall returns. They are 1) transplanting of feminized seedlings (clones or feminized seed germinated indoors), 2) feminized seed planted directly into the field, and 3) row crop production of dioecious variety with elevated CBD content. Costs have been compared for the three methods (FIGS. 5A-D) using the Hemp & Enterprise CBD Budget Model referenced in the University of Kentucky, March 2019 Economic & Policy Update (Volume 19, Issue 3 titled “The Economics of Hemp Production in Kentucky” (Shepherd J and Mark T, 2019; hemp.ca.uky.edu).

A few major differences among the model inputs include the total CBD content of the variety (Table 7), income from hemp grain (not a product in feminized production), seed costs (because dioecious seed can be created outdoors at lower costs), labor costs (for rogueing males, weeding, etc.; hempbenchmarks.com), lab testing (it is common practice for growers to test many times to time harvest before the crop exceeds 0.30% THC; see Table 7) and interest on operating capital (because overall input costs are higher, and the average flowering/maturity time of current genetics are later; see Table 7).

Table 7 shows typical cannabinoid content estimates as determined by High-performance liquid chromatography (% dry wt) in female bud tissue harvested from a high CBD NWG proprietary line and thirteen comparator varieties purchased on the open market. Varieties 11 thru 13 are certified varieties bred for grain production in France.

TABLE 7

| | | Avg | Avg | Days to |
|---|---|---|---|---|
| | Genotype | THC | CBD | Flower |
| NWG | NWG Proprietary line | 0.26 | 6.15 | 55 |
| 1 | Cherry Blossom | 0.34 | 1.92 | 73 |
| 2 | T-1 #5/Abacus-2-M | 0.42 | 8.97 | ≥74 |
| | Abacus-2-M/Cherry | | | |
| 3 | Wife | 0.81 | 8.20 | ≥74 |
| 4 | Abacus-2-M | 0.40 | 9.45 | ≥74 |
| 5 | Stormy Daniels | 0.42 | 8.09 | ≥74 |
| 6 | Otto II/BaOx | 0.08 | 1.56 | 66 |
| 7 | Cherry 5 | 0.28 | 6.65 | ≥74 |
| 8 | Stable Fire Cherry | 0.37 | 7.39 | ≥74 |
| 9 | Cherry 307 | 0.37 | 7.23 | ≥74 |
| 10 | Cherry Blossom | 0.36 | 8.22 | ≥74 |
| 11 | Earlina08FC | 0.00 | 0.71 | 39 |
| 12 | Fedora 17 | 0.00 | 0.96 | 55 |
| 13 | Ferimon 12 | 0.00 | 0.85 | 56 |

The NWG proprietary line has elevated levels of cannabidiol (CBD; 5% or greater). Table 8 shows typical cannabinoid content estimates in female bud tissue harvested from the NWG proprietary line.

TABLE 8

| THC | THC- | THC- | CBD | CBD- | CBD- | CBN | CBG | |
|---|---|---|---|---|---|---|---|---|
| % | A % | total | % | A % | total | % | total | CBD:THC |
| 0.00 | 0.30 | 0.26 | 0.00 | 6.89 | 6.15 | 0.00 | 0.31 | 23.55 |

### Deposits

Applicant(s) made a deposit of at least 2500 seeds of hemp Cannabis cultivars NWG331 and NWG452 with an International Depositary Authority as established under the Budapest Treaty according to 37 CFR 1.803(a)(1), at the National Collections of Industrial, Food and Marine Bacteria Ltd. (NCIMB) in Aberdeen Scotland, Accession No. NCIMB 43290 and NCIMB 43280. The NWG331 and NWG452 seeds deposited therewith on Nov. 23, 2018 and Nov. 22, 2018, respectively, were taken from the deposit maintained by New West Genetics, PO Box 1662 Fort Collins, Colo. 80522 since prior to the filing date of this application. Access to this deposit will be available during the pendency of the application to the Commissioner of Patents and Trademarks and persons determined by the Commissioner to be entitled thereto upon request. Upon issue of claims, the Applicant(s) will make available to the public, pursuant to 37 CFR 1.808, a deposit of at least 2500 seeds of cultivar NWG331 and NWG452 with an International Depositary Authority as established under the Budapest Treaty according to 37 CFR 1.803(a)(1), at the National Collections of Industrial, Food and Marine Bacteria Ltd. (NCIMB) in Aberdeen Scotland.

This deposit will be maintained in the depository, which is a public depository, for a period of 30 years, or 5 years after the most recent request, or for the enforceable life of the patent, whichever is longer, and will be replaced if it becomes nonviable during that period. Additionally, Applicants have or will satisfy all the requirements of 37 C.F.R. §§ 1.801-1.809, including providing an indication of the viability of the sample. Applicants have no authority to waive any restrictions imposed by law on the transfer of biological material or its transportation in commerce. Applicants do not waive any infringement of their rights granted under this patent or under the Plant Variety Protection Act (7 USC 2321 et seq.).

What is claimed is:

1. A method of increasing Cannabinoid yields from Cannabis comprising:
planting seeds from a Cannabis variety with an average CBD content of greater than about 1% by weight of female bud tissue;
allowing seeds to grow into Cannabis plants with female buds;
harvesting said Cannabis plants; and thereafter
extracting CBD from said harvested Cannabis bud tissue.

2. The method of claim 1, wherein said Cannabis is grown at large-scale commercial row crop production outdoors.

3. The method of claim 1, wherein said Cannabis variety exhibits an average CBD content of greater than 1.07% by weight of female bud tissue.

4. The method of claim 1, wherein said variety has an average CBD content of 2.54 wt. % in female bud tissue.

5. The method of claim 1, wherein said ratio of CBD:THC is 56:1.

6. The method of claim 1, wherein said Cannabis variety produces female buds with an average CBD content of about 2.83%.

7. The method of claim 1, wherein said THC content is zero.

8. The method of claim 1, wherein said extraction is by super-critical CO₂, hydrocarbon (propane, butane, or other), ethanol or steam distillation.

9. The method of claim 1, wherein said extraction does not require THC separation.

10. The method of claim 1, wherein said Cannabis variety medium to short plant height.

11. The method of claim 1, wherein said Cannabis variety produces plants with reduced branching.

12. The method of claim 1, wherein said Cannabis variety produces plants with an average plant height of about 170 cm to 190 cm.

13. The method of claim 1, wherein said Cannabis variety produces plants with a middle third of the plant characterized by medium stem internode length.

14. The method of claim 1, wherein said variety has medium branching.

15. The method of claim 1, wherein said Cannabis is planted at a higher than average density to increase CBD per acre.

16. The method of claim 1, wherein said Cannabis variety is NWG331.

17. The method of claim 1, wherein said Cannabis variety is NWG452.

18. The method of claim 1, wherein said Cannabis variety exhibits increased cannabinoid preservation.

19. The method of claim 18, wherein the increased cannabinoid preservation is due to trichomes resistant to damage from physical contact, heat, light, and/or oxygen.

20. The method of claim 1, wherein said seed is not feminized seed.

21. The method of claim 1, wherein the method further comprises harvesting seed from said Cannabis plants.

22. A method of increasing CBD content in a Cannabis variety comprising:
a) crossing a plant from a variety with a CBD content of greater than 2% by weight in female bud tissue with a plant of another Cannabis cultivar in which is desired to increase CBD content to produce progeny plants;
b) identifying progeny plants with increased CBD content in female bud tissue;
c) selecting one or more progeny plants with increased CBD content to produce selected progeny plants;
d) repeating steps (a) through (c) to as necessary to produce selected progeny plants that comprise the CBD level in a progeny line.

23. A Cannabis progeny line produced by the method of claim 22.

24. The method of claim 22, wherein said Cannabis variety with greater than 1% of CBD exhibits and average CBD content of greater than 2% by weight of female bud tissue.

25. The method of claim 22, wherein said Cannabis variety with greater than 2% of CBD variety has an average CBD content of 2.54 wt. % in female bud tissue.

26. The method of claim 22, wherein said Cannabis variety with greater than 2% of CBD produces female buds with an average CBD content of about 2.83 or higher.

27. The method of claim 22, wherein said plants are selected for medium to short plant height.

28. The method of claim 22, wherein said Cannabis variety with greater than 2% of CBD produces plants with an average plant height of about 170 cm to 190 cm.

29. The method of claim 22, wherein said plants are selected for reduced branching.

30. The method of claim 22, wherein said Cannabis variety with greater than 2% of CBD produces plants with a middle third of the plant characterized by medium stem internode length.

31. The method of claim 22, wherein said Cannabis variety with greater than 2% of CBD said has medium branching.

32. The method of claim 22, wherein said progeny Cannabis may be planted at a higher than average density to increase CBD per acre.

33. The method of claim 22, wherein said Cannabis variety with greater than 2 wt. % of CBD in female bud tissue is NWG331.

34. The method of claim 22, wherein said Cannabis variety with greater than 2 wt. % of CBD in female bud tissue is NWG452.

35. A method of increasing Cannabinoid yield per acre comprising:
planting seeds of the Cannabis variety produced by the method of claim 15;
allowing said seeds to grow into Cannabis plants;
harvesting said plants and thereafter extracting Cannabinoids female bud tissue from said harvested plants.

36. The method of claim 35, wherein said extraction is by super-critical CO₂, hydrocarbon (propane, butane, or other), ethanol or steam distillation.

37. The method of claim 35, wherein said extraction does not require THC separation.

38. A Cannabis variety having increased cannabinoid preservation.

39. The Cannabis variety of claim 38, wherein the increased cannabinoid preservation is due to trichomes resistant to damage from physical contact, heat, light, and/or oxygen.

40. The Cannabis variety of claim 38, wherein the increased cannabinoid preservation is due to the plant architecture.

41. The Cannabis variety of claim 38, wherein the increased cannabinoid preservation is due to the higher trichome density.

42. The Cannabis variety of claim 38, wherein the increased cannabinoid preservation is due to the specific morphological characteristics.

43. The Cannabis variety of claim 39, wherein at least 60%, at least 70%, at least 80%, at least 90%, at least 95%, at least 98%, or at least 99% of the trichomes are preserved during mechanized harvest.

44. A method for large-scale commercial production of cannabinoids comprising:
planting seeds of a Cannabis variety having increased cannabinoid preservation;
allowing seeds to grow into Cannabis plants with female buds; and
harvesting said Cannabis plant, or a plant part thereof.

45. The method of claim 44, wherein the plant part is female chaff, buds or flowers.

46. The method of claim 44, wherein the trichomes of said Cannabis plants are resistant to damage from physical contact, heat, light, and/or oxygen.

47. The method of claim 44, wherein the increased cannabinoid preservation is due to plant architecture.

48. The method of claim 44, wherein the increased cannabinoid preservation is due to higher trichome density.

49. The method of claim 44, wherein the increased cannabinoid preservation is due to specific morphological characteristics.

50. The method of claim 44, wherein said harvesting is with a mechanized harvester.

51. The method of claim 50, wherein at least 60%, at least 70%, at least 80%, at least 90%, at least 95%, at least 98%, or at least 99% of the trichomes are preserved during mechanized harvest.

52. The method of claim 44, wherein the seeds are planted outdoors.

53. The method of claim 44, wherein said Cannabis is planted at a higher than average density.

54. The method of claim 44, wherein the planting rate is greater than 14 pounds per acre.

55. The method of claim 44, further comprising:
extracting one or more cannabinoids from the harvested Cannabis plant, or a plant part thereof.

56. A cannabinoid composition produced from commercial production of the variety of claim 38.

57. The cannabinoid composition of claim 20, wherein the composition comprises one or more of THCV, CBDV, CBGV, CBCV, CBD, CBC, CBE, CBG, CBN, CBND, CBT, and their acid forms.

58. A method of developing a Cannabis variety with increased cannabinoid preservation comprising:
(a) crossing the plant of claim 38 with a second Cannabis plant to produce a progeny plant;
(b) crossing the progeny plant of step (a) with itself or the second Cannabis plant in step (a) to produce a seed;
(c) growing a progeny plant of a subsequent generation from the seed produced in step (b);
(d) crossing the progeny plant of a subsequent generation of step (c) with itself or the second Cannabis plant in step (a) to produce a Cannabis plant with increased cannabinoid preservation.

59. A Cannabis plant produced by the method of claim 58, said plant having increased cannabinoid preservation during mechanized harvest.

60. A method of assessing trichome and/or cannabinoid preservation of a Cannabis variety, the method comprising:
harvesting plant tissue from a plant of the Cannabis variety by hand and by machine;
extracting cannabinoids from the hand-harvested plant tissue and the machine-harvested plant tissue; and
comparing the abundance of cannabinoids in the hand-harvested plant tissue to the machine-harvested plant tissue.

61. The method of claim 60, further comprising:
selecting a Cannabis variety with increased trichome and/or cannabinoid preservation for large-scale commercial row crop production.

62. The method of claim 60, wherein the abundance is determined by gas chromatography (GC), high-performance liquid chromatography (HPLC) or near-infrared spectroscopy (NIR).

63. A method of conferring aroma, flavoring, or desired health benefits to a beverage comprising:
preparing said beverage with the Cannabis plant of claim 38, or parts thereof, or compositions purified therefrom.

64. The method of claim 63, wherein said beverage is beer, wine, cider, distilled spirit, hard soda, soft drink, juice, water, or flavored water.

65. A method of preparing cannabinoid isolates or isolate formulations, wherein the method comprises:
harvesting flower tissue from the plant of claim 38; and
extracting cannabinoids from the flower tissue.

66. A cannabis product produced or purified from the cannabis plant, or part thereof, of claim 38.

67. The cannabis product of claim 66, wherein the product is produced or purified from female chaff, buds or flowers.

68. The cannabis product of claim 66, wherein the product is a textile, a building material, a food or beverage, a personal hygiene product, a pharmaceutical or medicinal product, an industrial product, or an animal feed.
